(19) United States
(12) Patent Application Publication
(10) Pub. No.: US 2004/0259187 A1
(43) Pub. Date: Dec. 23, 2004

(54) Methods to measure lipid antioxidant activity

(76) Inventors: Giancarlo Aldini, Milan (IT); Kyung-Jin Yeum, Winchester, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/893,592

(22) Filed: Jul. 16, 2004

Related U.S. Application Data
(62) Division of application No. 10/114,181, filed on Apr. 2, 2002.
(60) Provisional application No. 60/280,920, filed on Apr. 2, 2001.

(51) Int. Cl. C12Q 1/26

(52) U.S. Cl. 435/25

(57) ABSTRACT

The present invention provides a selective method for measuring lipid antioxidant activity within a lipid compartment of a sample using lipophilic radical generators and oxidizable lipophilic indicators. The present invention accurately and efficiently determines the total antioxidant activity of a sample in both lipid and aqueous compartments. The methods of the invention can be used for diagnosing and protecting against disorders that arise from excess free radicals present in a subject. The reagents used in the methods of the invention can also be provided in a kit assay.

### RELATED APPLICATION

This application claims the benefit of U.S. Provisional Patent Application Ser. No. 60/280,920, filed Apr. 2, 2001, entitled “A Selective Method to Measure the Antioxidant Activity in the Aqueous and Lipid Compartments of Plasma,” the teachings of which are incorporated herein by reference.

### GOVERNMENT SUPPORT

This invention was made with government support under 58-1950-9-001 awarded by the United States Department of Agriculture. The government has certain rights in the invention.

### FIELD OF THE INVENTION

The present invention provides methods for a simple and efficient evaluation of lipid antioxidant activity. The invention also provides a method for determining the total antioxidant activity of a sample by accurately measuring the antioxidant activity of both the lipid compartment and the aqueous compartment.

### BACKGROUND OF THE INVENTION

Reduced levels of antioxidants have been linked to a number of pathological and disease states. Accordingly, it has been suggested that measurements of the oxidizability of biological samples from subjects can be useful to identify people at risk of developing a disease or disorder. Towards this end, studies have been conducted on the antioxidant activity of plasma (Ghiselli et al., Free Radic. Biol. Med. 18: 29-36 (1995); Cao et al., Clin. Chem. 41: 1738-44 (1995)), the antioxidant activity of synthetic and natural compounds (Murase et al., Free Radic. Biol. Med. 24: 217-25 (1998); Lotito et al., Free Radic. Biol. Med. 24: 435-441 (1998)), and the reactivity of hydrophilic and lipophilic antioxidants (Massaeli et al., Free Radic. Biol. Med. 26: 1524-30 (1999)). However, most of these methods rely upon a hydrophilic radical generator, 2,2′-azobis(2-amidinopropane) dihydrochloride (AAPH).

For the measurement of antioxidant capacity, the oxygen radical absorbance capacity, (ORAC) method is available. In the ORAC assay, AAPH is used as a radical initiator, and R-phycoerythrin is used as a probe, both of which are water-soluble compounds (Antolovich et al., Analyst 127: 183-198 (2002)). With the ORAC assay, although the ORAC values correlate with measured levels of water-soluble antioxidants (such as vitamin C and uric acid), there appears to be little, or no correlation between levels of fat-soluble antioxidants (such as carotenoids, tocopherols, and retinoids) and ORAC values (Cao et al. Free Rad. Biol. Med. 14: 303-311 (1993)).

Another available method is the total reactive antioxidant potential (TRAP) method. The TRAP assay uses AAPH as a radical initiator, and 2′,7′-dichlorodihydrofluorescein (DCFH) as a probe, both of which are water-soluble compounds. Again, the major antioxidants contributing to the TRAP value are water-soluble antioxidants such as uric acid, thiol groups, and protein, while fat-soluble antioxidants such as α-tocopherol contribute less than 5% of the TRAP value (Ghiselli et al., Free Rad. Biol. Med. 18: 29-36 (1995)). These existing methods for measuring total antioxidant capacity primarily use hydrophilic radical generators and hydrophilic probes, thereby limiting their measurement of the antioxidant capacity to the aqueous compartment of plasma. Thus, the total antioxidant activity of the sample is not accurately determined.

To measure the antioxidant activity of only the lipid compartment of a sample, current available methods rely on separating this compartment from the rest of the sample (Antolovich et al., Analyst 127: 183-198 (2002)). The process of separation causes unnecessary oxidation of the lipid compartment, resulting in artificial oxidation and contributing to the inaccuracy of the results obtained. In addition, the methods that rely on separating the lipid compartments require a large sample volume, large dilutions of the isolated lipid compartment, or the use of temperatures beyond a physiological range. All of these factors result in a sample that has deviated substantially from physiological conditions.

Accordingly, a need exists for methods that can selectively and accurately measure lipid compartment antioxidant activity under more physiological conditions. A need also exists for methods to measure the total antioxidant activity in samples.

### SUMMARY OF THE INVENTION

The present invention is based, in part, on the discovery of a method that selectively measures the lipid antioxidant activity within a lipid compartment of a sample. The invention relies on the use of lipophilic radical generators and oxidizable lipophilic indicators to determine the lipid antioxidant activity. The method of the invention can be used to accurately and efficiently determine the total antioxidant activity of a sample in both the lipid and aqueous compartments. Furthermore, the methods of the invention can be used for diagnosing and protecting against certain disorders that arise from oxidative stress and the presence of excess free radicals in a subject. The reagents used in the methods of the invention can also be provided in a kit assay.

Accordingly, in one aspect, the invention pertains to a method for measuring the lipid antioxidant activity in a sample by incubating the sample with a lipophilic radical generator at a concentration that produces free radicals in a lipid compartment of the sample. An oxidizable lipophilic indicator is also added to the sample, and the oxidation of the lipophilic indicator is measured to provide a measure of the antioxidant activity of the lipid compartment of the sample.

The sample can be a biological sample, such as blood, plasma, serum, cerebral spinal fluid, amniotic fluid, interstitial fluid, lymphatic fluid, synovial fluid, and tissue. In one embodiment, the sample is blood. In another embodiment, the sample is plasma.

The lipophilic radical generator can be a lipophilic radical generator that can generate free radicals in the lipid compartment of the sample at a level that can be readily measured. Suitable examples of lipophilic radical generators are azo radical generators that produce a flux of lipophilic radicals at a known constant rate. Other lipophilic radical generators may be organic hydroperoxides, such as cumene hydroperoxide and tert-butytl-hydroperoxide. Examples of azo radical generators include, but are not limited to, 2,2′-azobis(4-methoxy-2,4-dimethylvaleronitrile) (MeO-AMVN), 2,2′-azobis(2,4-dimethylvaleronitrile) (AMVN), azo-bis-isobutylnitrile, 2,2′-azobis(2-methylproprionate) (DAMP), 2,2′-azobis-(2-amidinopropane), and unsymmetrical azo initiators, such as 2,2′-azobis(2-amidinopropane)[2-(N-stearyl)amidinopropane], 2, 2′-azo[2-(2-imidiazolin-2-yl)-propane)-[2-[2-(4-n-octyl)imidazolin-2-yl]-propane] (Culbertson et al., Free Radic. Res. 33(6): 705-718 (2001)). In a preferred embodiment, the lipophilic radical generator is 2,2′-azobis(4-methoxy-2,4-dimethylvaleronitrile) (MeO-AMVN).

The antioxidant activity of the lipid compartment is detected using an oxidizable lipophilic indicator such as a fluorescent probe that is responsive to lipid oxidation. Examples of fluorescent probes include, but are not limited to boron-containing fluorogenic probes, such as boron dipyrromethene difluoride (BODIPY), 4,4-difluoro-3a,4a-diaza-s-indacene (BODIPY) fatty acids, known as “BDY” fatty acids, pyrene fatty acid derivatives, perlene fatty acids, cis-parinaric acid, diphenyl-1-pyrenylphosphine (DPPP), and lipophilic fluorescein dyes. In a one embodiment, the oxidizable lipophilic indicator is a BODIPY fatty acid selected from the group consisting of BODIPY 576/589, BODIPY 581/591, and BODIPY 665/676 (Molecular Probes, Eugene, Oreg.). In a preferred embodiment, the BODIPY fatty acid is BODIPY 581/591. The step of measuring the oxidation of the oxidizable lipophilic probe provides an indirect measurement of antioxidant activity of the lipid compartment of the sample.

The present invention can also be used to accurately and efficiently determine the total antioxidant activity of a sample by accurately measuring the antioxidant activity of both the lipid and aqueous compartments. Accordingly, in another aspect, the invention pertains to a method for measuring the total antioxidant activity in a sample by incubating the sample with a lipophilic radical generator at a concentration that produces free radicals in a lipid compartment of the sample, and a hydrophilic radical generator at a concentration that produces free radicals in an aqueous compartment of the sample. An oxidizable lipophilic indicator, and an oxidizable hydrophilic indicator are also added to the sample. The oxidation of the lipophilic indicator is measured to provide a measure of the antioxidant activity of the lipid compartment of the sample, and the oxidation of the hydrophilic indicator is measured to provide a measure of the antioxidant activity of the aqueous compartment of the sample.

In one embodiment, the antioxidant activity is measured in one sample that has the lipophilic radical generator, the oxidizable lipophilic indicator, the hydrophilic radical generator, and the oxidizable hydrophilic indicator. In another embodiment, the antioxidant activity is measured in at least two separate samples. The first sample has the lipophilic radical generator and the oxidizable lipophilic indicator, while the second sample has the hydrophilic radical generator and the oxidizable hydrophilic indicator. The total antioxidant activity is measured by combining the results from each of the separate samples.

Examples of lipophilic radical generators are described above. In a preferred embodiment, the lipophilic radical generator is 2,2′-azobis(4-methoxy-2,4-dimethylvaleronitrile) (MeO-AMVN). The sample can be incubated with a hydrophilic radical generator that includes, but is not limited to, an azo radical generator, 2,2′-azobis[2-(5-methyl-2-imidazolin-2-yl)propane]dihydrochloride, iron, ascorbic acid and metal ions. In one embodiment, the hydrophilic radical generator is an azo radical generator selected from the group consisting of 2,2′ azobis(2-amidinopropane)dihydrochloride (AAPH), and unsymmetrical azo initiators, such as 2,2′-azobis(2-amidinopropane)[2-(N-stearyl)amidinopropane]dihydrochloride (SA-1), 2,2′-azo[2-(2-imidiazolin-2-yl)-propane)-[2-[2-(4-n-octyl)imidazolin-2-yl]-propane]dihydrochloride (C-8). In a preferred embodiment, the hydrophilic radical generator is 2,2′ azobis(2-amidinopropane)dihydrochloride (AAPH).

The antioxidant activity of the lipid compartment can be measured using an oxidizable lipophilic indicator that is responsive to lipid oxidation, while the antioxidant activity of the aqueous compartment can be measured using an oxidizable hydrophilic indicator that is responsive to aqueous oxidation. Examples of oxidizable lipophilic are described above. Examples of oxidizable hydrophilic indicators that are responsive to aqueous oxidation can also be fluorescent probes that include, but are not limited to dichlorodihydrofluorescein (DCFH), BODIPY FL EDA, and BODIPY FL hexadecanoic acid. The step of measuring the oxidation of the oxidizable lipophilic indicator provides an indirect measurement of antioxidant activity of the lipid compartment of the sample, while the step of measuring the oxidation of the oxidizable hydrophilic indicator provides an indirect measurement of antioxidant activity of the aqueous compartment of the sample.

In another aspect, the invention pertains to a method of diagnosing a free radical associated disorder, or oxidative stress in a subject, by measuring the level of lipid antioxidant activity in a sample from a subject. For example, normal range of antioxidant capacity in the lipid compartment can be determined statistically from the data obtained by analyses of fat-soluble antioxidant levels, such as carotenoids and tocopherols, and lipophilic antioxidant capacity in a large population of healthy individuals. The measured activity of the lipid antioxidant is compared with at least one known normal value for the lipid antioxidant to determine whether a deviation from the normal value exists. The known normal value (or range of values) can be determined using standard techniques. For example, total antioxidant levels can be determined for a large population of healthy individuals and normal ranges can be statistically determined.

In one embodiment, the level of lipid antioxidant activity is a measure of the entire lipid composition i.e., all the lipid components in the lipid compartment. In another embodiment, the level of lipid antioxidant activity is a measure of a fraction of the lipid composition, e.g., the LDL component of the lipid compartment, or the VLDL component of the lipid compartment.

In another embodiment, the aqueous antioxidant activity of a sample can be determined to diagnose a free radical associated disorder or oxidative stress by measuring the level of an aqueous antioxidant activity in a sample from a subject. The measured activity of the aqueous antioxidant is compared with at least one known normal value of the aqueous antioxidant to determine whether a deviation from the normal value exists. Normal range of antioxidant capacity in aqueous compartment can be determined statistically from the data obtained by analyses of water-soluble antioxidant levels, such as ascorbic acid, uric acid and water-soluble flavonoids (catechin, epigallocatechin gallate etc.), and hydrophilic antioxidant capacity in a large population of healthy individuals.

In another embodiment, the total antioxidant activity of a sample can be determined by combining the measured level of the lipid antioxidant activity and the measured level of the aqueous antioxidant activity.

In another aspect, the invention pertains to a method of protecting against a free radical associated disorder, or oxidative stress, by identifying a reduced lipid antioxidant activity in the lipid compartment of a sample from a subject, and administering a lipid antioxidant at a concentration that increases the lipid antioxidant concentration in the lipid compartment, such that the increase of lipid antioxidant in the lipid compartment protects against the free radical associated disorder or oxidative stress. In one embodiment, at least one lipid antioxidant is administered, e.g., α-tocopherol. In another embodiment, a combination of lipid antioxidants are administered, e.g., α-tocopherol and carotenoids such as lutein, lycopene and β-carotene.

In another embodiment, the method of protecting may further involve identifying a reduced aqueous antioxidant activity in the aqueous compartment of a sample from a subject, and administering an aqueous antioxidant at a concentration that increases the aqueous antioxidant concentration in the aqueous compartment, such that the increase of aqueous antioxidant in the aqueous compartment protects against the free radical associated disorder or oxidative stress.

In one embodiment, at least one aqueous antioxidant is administered, e.g., ascorbic acid. In another embodiment, a combination of aqueous antioxidants are administered, e.g., ascorbic acid and water-soluble polyphenols such as catechins, isoflavones, and procyanidins. Uric acid may be increased by ingesting uric acid containing food, and polyphenols. In yet another embodiment, at least one aqueous antioxidant e.g., ascorbic acid and at least one lipid antioxidant, e.g., α-tocopherol are administered. In yet another embodiment, a combination of aqueous antioxidants e.g., ascorbic acid and water-soluble polyphenols such as catechins, isoflavones, and procyanidins, and ascorbic acid and combination of lipid antioxidants, e.g., α-tocopherol and β-carotene are administered.

In another aspect, the invention pertains to a method of assessing the efficacy of a therapy for a free radical associated disorder or oxidative stress by measuring the lipid antioxidant activity in a sample from a subject, and measuring the lipid antioxidant activity in a second sample obtained from the subject following the therapy. A higher lipid antioxidant activity in the second sample compared to the first sample, is an indication that the therapy is efficacious for the free radical associated disorder or oxidative stress.

In one embodiment, the method further comprises measuring the aqueous antioxidant activity in a sample from a subject, and measuring the aqueous antioxidant activity in a second sample obtained from the subject following the therapy. A higher aqueous antioxidant activity in the second sample compared to the first sample, is an indication that the therapy is efficacious for the free radical associated disorder or oxidative stress.

In another aspect, the invention pertains to an assay kit comprising a lipophilic radical generator capable of producing free radicals in a lipid compartment of the sample, and an oxidizable lipophilic indicator capable of providing a measure of antioxidant activity in the lipid compartment of the sample. In one embodiment, the assay kit further comprises a hydrophilic radical generator capable of producing free radicals in an aqueous compartment of the sample, and an oxidizable hydrophilic indicator capable of providing a measure of antioxidant activity in the aqueous compartment of the sample.

### BRIEF DESCRIPTION OF THE DRAWINGS

FIG. 1 is a graph comparing the effects of AAPH and MeO-AMVN on the levels of the hydrophilic antioxidants ascorbic acid (AA) and uric acid (UA) in human plasma over time;

FIG. 2A is a graph comparing the effect of AAPH and MeO-AMVN on the level of the lipophilic antioxidant α-tocopherol in human plasma over time;

FIG. 2B is a graph comparing the effect of AAPH and MeO-AMVN on the level of the lipophilic antioxidant β-carotene in human plasma over time;

FIG. 3 is a graph comparing the oxidation of DCFH to DCF induced by AAPH or MeO-AMVN over time;

FIG. 4 is a graph of a time-course showing the development of red and green fluorescence from the addition of C11-BODIPY 581/591 in the presence of AMVN or MeO-AMVN in human plasma;

FIG. 5 is a graph of a time-course of BODIPY green fluorescence in human plasma in the presence of MeO-AMVN or AAPH;

FIG. 6 is a bar graph showing the effect on lipid plasma oxidizability induced by MeO-AMVN and measured using BODIPY for pre-incubation time of human plasma with the lipophilic antioxidant α-tocopherol or β-carotene;

FIG. 7A is a graph showing EGCG inhibition of aqueous plasma compartment oxidation induced by AAPH and monitored by DCF fluoroscence over time;

FIG. 7B is a graph showing the effect of EGCG on lipid plasma compartment oxidation induced by MeO-AMVN and monitored by measuring BODIPY green fluorescence over time;

FIG. 8 is a bar graph depicting the dose-dependent protective effect of EGCG on aqueous and lipid compartment oxidation after 180 min of incubation;

FIG. 9 is a bar graph depicting the dose-dependent effect of EGCG on α-tocopherol depletion induced by AAPH and MeO-AMVN;

FIG. 10 is an ESR spectra time-course of α-TOC-O. decay in absence (A) and presence (B) of EGCG;

FIG. 11 is a graphical depiction of the proposed antioxidant mechanism of EGCG in human plasma;

FIG. 12 is a graph showing the direct correlation of a high lycopene diet on the lipid oxidizability monitored via the production of green fluorescence from BODIPY;

FIG. 13 is a graph showing the effect of BHT on lipid oxidizability of plasma; and

FIG. 14 is a graph showing the effect the time of preincubation with BHT on the lipid oxidizability of plasma.

### DETAILED DESCRIPTION OF THE INVENTION

The methods of the invention can be used to selectively and accurately measure the lipid antioxidant activity within the lipid compartment of a sample using lipophilic radical generators and lipophilic initiators. The methods of the invention can also be used to measure the total antioxidant activity of a sample by accurately determining the antioxidant activity of both the lipid and the aqueous compartments of a sample. The practice of the present invention employs, unless otherwise indicated, conventional methods of sample isolation, redox chemistry and spectroscopy.

So that the invention is more clearly understood, the following terms are defined:

The term “antioxidant” as used herein refers to a substance that, when present in a mixture or structure containing an oxidizable substrate molecule (e.g., an oxidizable biological molecule or oxidizable indicator), significantly delays or prevents oxidation of the oxidizable substrate molecule. Antioxidants can act by scavenging biologically important reactive free radicals or other reactive oxygen species (e.g., O₂⁻, H₂O₂, HOCl, ferryl, peroxyl, peroxynitrite, and alkoxyl), or by preventing their formation, or by catalytically converting the free radical or other reactive oxygen species to a less reactive species. Antioxidants can be separated into two classes, lipid antioxidants, and aqueous antioxidants. Examples of lipid antioxidants include, but are not limited to, carotenoids (e.g. lutein, zeaxanthin, β-cryptoxanthin, lycopene, α-carotene, and β-carotene), which are located in the core lipid compartment, and tocopherols (e.g. vitamin E, α-tocopherol, γ-tocopherol, and δ-tocopherol), which are located in the interface of the lipid compartment, and retinoids (e.g. vitamin A, retinol, and retinyl palmitate) and fat-soluble polyphenols such as quercetin. Examples of aqueous antioxidants include, but are not limited to, ascorbic acid and its oxidized form, “dehydroascorbic acid”, uric acid and its oxidized form, “allantoin”, bilirubin, albumin and vitamin C and water-soluble polyphenols such as catechins, which have high affinity to the phospholipid membranes, isoflavones, and procyanidins.

When one more antioxidants are added to a test sample or assay, a detectable decrease in the amount of a free radical, such as superoxide, or a nonradical reactive oxygen species, such as hydrogen peroxide, may be seen in the sample, compared with a sample untreated with the antioxidant (i.e. control sample) or assay reaction. Electron spin resonance (ESR) can be used to measure free radicals directly. However, numerous indirect methods exist such as monitoring the change in antioxidant status, assays that trap hydroxyl radicals, and monitoring degradation products caused by free radicals (i.e. lipid peroxidation). Suitable concentrations of antioxidants measured to produce the desired change or amelioration, (e.g., an efficacious or therapeutic dose) can be determined by various methods, including generating an empirical dose-response curve.

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact-molecules, they abstract a part to complete their own electronic structure, generating new radicals, which go on to react with other molecules. Such chain reactions are particularly important in decomposition of substances at high temperatures and in polymerization. In the body, oxidized (see oxidation-reduction) free radicals can damage tissues. Antioxidant nutrients (e.g., vitamins C and E, selenium, polyphenols) may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO.), singlet oxygen (¹O₂), superoxide anion radical (O.₂⁻), nitric oxide radical (NO.), peroxyl radical (ROO.), peroxynitrite (ONOO⁻) can be in either the lipid or compartments.

The phrase “lipid compartment” as used herein refers to members of a class of compounds that contain cyclic or acyclic long-chain aliphatic hydrocarbons and their derivatives, such as fatty acids, alcohols, amines, amino alcohols, and aldehydes. By way of non-limiting example, common lipids include fatty acids, fats, phospholipids, steroids, eicosanoids, waxes, and fat-soluble vitamins. Some lipids may be generally classified into two groups, the simple lipids and the complex lipids. By way of non-limiting example, simple lipids include triglycerides or fats and oils, which are fatty acid esters of glycerol, waxes, which are fatty acid esters of long-chain alcohols, and steroids such as cholesterol and ergosterol, which are derived from partially or completely derived pheanthrene. Complex lipids include, for example, phosphatides or phospholipids, which are lipids that contain phosphorous, glycolipids, which are lipids that contain carbohydrate residues, and sphingolipids, which are lipids containing sphingosine, a long-chain alcohol.

The term “lipid” includes fats or fat-like substances. The term is descriptive rather than a chemical name such as protein or carbohydrate. Lipids include true fats (i.e., esters of fatty acids and glycerol), lipoids (i.e., phospholipids, cerebrosides, waxes) and sterols (i.e., cholesterol, ergostrol). Lipids can be a target of oxidation through mechanisms, such as autoxidation.

The term “fatty acid” as used herein refers to a group of negatively charged, generally linear hydrocarbon chains. The hydrocarbon chains of fatty acids vary in length and oxidation states. Each fatty acid has a negatively charged portion, which is located at a carboxyl end group, and a “tail” portion, which determines the water solubility and amphipathic characteristics of the fatty acid. By way of non-limiting example, fatty acids can be found as components of the phospholipids that comprise biological membranes, as fats, which are used to store energy inside cells, or as a means for transporting fat in the bloodstream.

The term “phospholipid” as used herein refers to any of the class of esters of phosphoric acid that contain at least one molecule of fatty acid, an alcohol, and a nitrogenous base.

The term “fats” as used herein refer to the any of the glyceryl esters of fatty acids, for example, the monoacylglycerol, diacylglycerol and triacylglycerol forms of fatty acids. Triglycerides refer to those molecules that are neutrally charged and entirely hydrophobic, i.e., reduced molecules. Monoacylglycerides and diacylglycerides are metabolic intermediates in phospholipid synthesis, while triglycerides form the fat molecules that are used to store chemical energy in a water free, compact state.

The term “steroids” as used herein refers to a member of a group of compounds that are derived or partially derived from cyclopenat[α]-phenanthrene, which is a fused, reduced ring system that consists of three fused cyclohexane rings in a non-linear or phenanthrene arrangement. Steroids can be used as signaling molecules that readily diffuse across biological membranes. By way of non-limiting example, steroids can be hormonal steroids, for example testosterone and progesterone, or they can be non-hormonal steroids, for example cholesterol and compounds that are derived from cholesterol, for example ergosterol and cholic acid.

The term “eicosanoids” as used herein refers to any of the specialized fatty acid derivatives that are derived from polyunsaturated fatty acids. Eicosanoids are commonly found in cell membranes. The two major groups of eicosanoids include prostaglandins and leukotrienes.

The term “fat-soluble vitamins” as used herein refers to any member of the mixed group of linear and cyclic π-electron systems. By way of non-limiting example, common fat-soluble vitamins include vitamin (A) (retinol) and vitamin D₃ (cholecalciferol).

The phrase “lipid antioxidant activity” or “lipid antioxidant capacity” are used interchangeably herein and refer to the measurement of antioxidant ability arising from the lipid compartment of a sample.

The phrase “aqueous compartment” as used herein refers the portion of a fluid sample that does not form the lipid compartment. The aqueous compartment can be a biological fluid sample for example, blood, plasma, serum, cerebral spinal fluid, amniotic fluid, interstitial fluid; lymphatic fluid, and synovial fluid. By way of non-limiting example, the aqueous compartment of a fluid sample such as serum may include not only the liquid portion that remains after blood has been allowed to clot and is centrifuged to remove the blood cells and clotting elements, but also other compounds such as: proteins, e.g., albumin and globulins; antibodies; enzymes; small amounts of nutritive organic materials, such as amino acids and glucose; inorganic substances such as sodium, choloride, sulfates, phosphates, calcium, potassium, bicarbonate, magnesium, iodine, zinc, and iron; small amounts of waste products, such as urea, uric acid, xanthine, creatinine, creatine, bile pigments and ammonia; and trace amounts of gases such as oxygen and carbon dioxide. The fluid sample may also be a non-biological sample, for example, chemical formulations, synthetic compositions, or food products and cosmetic products.

The phrase “aqueous antioxidant activity” or “aqueous antioxidant capacity” are used interchangeably herein and refer to the measurement of antioxidant ability arising from the aqueous compartment of the sample.

The phrase “total antioxidant activity” or “total antioxidant capacity” are used interchangeably herein and refer to the combined antioxidant ability arising from the aqueous compartment and lipid compartment.

The term “sample” as used herein refers to a test item that has at least one compartment in which free radicals can be generated using a free radical generator, (e.g., a lipophilic free radical generator or an hydrophilic free radical generator) and can be detected with an indicator, e.g., an oxidizable lipophilic indicator, or an oxidizable hydrophilic indicator). The sample can be a liquid or fluid biological sample, or a solid biological sample. The biological sample can be a liquid sample e.g., blood, plasma, serum, cerebral spinal fluid, urine, amniotic fluid, interstital fluid, and synovial fluid. The sample may be a solid e.g., a tissue or cell matter. The term sample also refers to as a non-biological sample such as a chemical solution, synthetic composition, and food.

The phrase “lipophilic radical generator” or “lipophilic radical initiator” are used interchangeably herein and refer to an agent, compound, or molecule that can produce free radicals in the lipid compartment of a sample. The lipophilic radical generator should be capable of producing free radicals at a measured level, for example, at a level at which antioxidants or oxidizable indicators can interact with the free radicals to produce a measurable or detectable output. Examples of lipophilic radical generator are described below.

The phrase “hydrophilic radical generator” or “hydrophilic radical initiator” are used interchangeably herein and refer to an agent, compound, molecule that can produce free radicals in the aqueous compartment of a sample. The hydrophilic radical generator should be capable of producing free radicals at a measured level, for example, at a level at which antioxidants or oxidizable indicators can interact with the free radicals to produce a measurable or detectable output. Examples of hydrophilic radical generator are described below.

The phrase “oxidizable lipophilic indicator” as used herein refers to a lipid soluble indicator that interact with a lipid free radical and becomes oxidized. The change in state of the lipid indicator from a non-oxidized to an oxidized state can be monitored directly (e.g., fluorescent color change of BODIPY) or indirectly (e.g., consumption of antioxidants; the free radicals that are scavenged by the antioxidant are no longer available to oxidize the oxidizable lipid indicator). Examples of oxidizable lipid indicators include, but are not limited to, BODIPY fatty acids, pyrene fatty acid derivatives, perlene fatty acids, cis-parinaric acid, diphenyl-1-pyrenylphosphine (DPPP), hexadecanamide, N-(3′,6′-dihydroxy-3-oxospiro(isobenzofuran-1(3H),9′-(9H)xanthen)-5-yl), and lipophilic fluorescein dyes.

The phrase “oxidizable hydrophilic indicator” as used herein refers to an aqueous soluble indicator that interacts with an aqueous free radical and becomes oxidized. The change in state of the aqueous indicator can be monitored directly (e.g., fluorescent color change of BODIPY) or indirectly (e.g., consumption of antioxidants; the free radicals that are scavenged by the antioxidant are no longer available to oxidize the oxidizable lipid indicator). Examples of oxidizable hydrophilic indicators include, but are not limited to, dichlorodihydrofluorescein (DCFH), R-phycoerythrin, 4,4-difluoro-5,7-dimethyl-4-bora-3a,4a-diaza-s-indacene-3-propionyl ethylenediamine, hydrochloride, BODIPY FL EDA, and BODIPY FL hexadecanoic acid.

The phrase “azo radical generator” as used herein refers a class of compounds that produce a flux of free radicals at a known constant rate. Examples of lipophilic azo radical generators include, but are not limited to, 2,2′-azobis(4-methoxy-2,4-dimethylvaleronitrile) (MeO-AMVN), 2,2′-azobis(2,4-dimethylvaleronitrile) (AMVN), azo-bis-isobutylnitrile, 2,2′-azobis(2-methylproprionate) (DAMP), and 2,2′-azobis-(2-amidinopropane). Examples of hydrophilic azo radical generators include, but are not limited to, 2,2′-azobis[2-(5-methyl-2-imidazolin-2 yl)propane]dihydrochloride, iron, ascorbic acid and metal ions.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The phrase “free radical associated disorder” as used herein refers to a pathological condition of in a subject that results at least in part from the production of or exposure to free radicals, for example, oxyradicals, or other reactive oxygen species in vivo. The term “free radical associated disorder” encompasses pathological states that are recognized in the art as being conditions wherein damage from free radicals is believed to contribute to the pathology of the disease state, or wherein administration of a free radical inhibitor (e.g., desferrioxamine), scavenger (e.g., tocopherol, glutathione), or catalyst (e.g., SOD, catalase) are shown to produce a detectable benefit by decreasing symptoms, increasing survival, or providing other detectable clinical benefits in protecting or preventing the pathological state. Examples of free radical disorders include, but are not limited to, ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, age-related macular degeneration, Alzheimer's disease, uveitis, emphysema, gastric ulcers, oxygen toxicity, neoplasia, undesired cell apoptosis, and radiation sickness. Such diseases can include “apoptosis-related ROS” which refers to reactive oxygen species (e.g., O₂⁻) which damage critical cellular components (e.g., lipid peroxidation) in cells stimulated to undergo apoptosis, such apoptosis-related ROS may be formed in a cell in response to an apoptotic stimulus and/or produced by non-respiratory electron transport chains (i.e., other than ROS produced by oxidative phosphorylation).

The term “oxidative stress” as used herein refers to the level of damage produced by oxygen free radicals in a subject. The level of damage depends on how fast reactive oxygen species are created and then inactivated by antioxidants.

The term “deviation” or “deviate” are used interchangeably herein and refer to a change in the antioxidant activity of a sample. The change can be an increase, decrease, elevation, or depression of antioxidant activity from a known normal value. For example, a increase or decrease of antioxidant activity in the lipid compartment of a sample, the aqueous compartment of a sample, or in both the lipid and aqueous compartment of the sample.

The invention is described in more detail in the following subsections:

I Isolation of Samples

One aspect of the invention pertains to a method for measuring lipid antioxidant activity in sample by using lipophilic radical generators and oxidizable lipophilic indicators. The sample can be isolated using standard techniques. If the sample is a biological fluid, e.g., blood, it can be extracted from a subject using a syringe using known techniques. The method of the present invention is suitable for use on any other type of sample fluid (e.g., serum, plasma, cerebral spinal fluid, amniotic fluid, synovial fluid, interstitial fluid. The sample may also be a solid such as a tissue or cell matter. The tissue sample may first need to be solubilized or fractionated using standard techniques known in the art, such as enzymatic lysis and French pressing.

In order to isolate the lipid compartment from the sample, e.g., blood sample, standard techniques such as centrifugation can be used. The lipid compartment comprises compounds that contain cyclic or acyclic long-chain aliphatic hydrocarbons and their derivatives, such as fatty acids, alcohols, amines, amino alcohols, and aldehydes. By way of non-limiting example, common lipids include fatty acids, fats, phospholipids, steroids, eicosanoids, waxes, and fat-soluble vitamins. Some lipids may be generally classified into two groups, the simple lipids and the complex lipids. By way of non-limiting example, simple lipids include triglycerides or fats and oils, which are fatty acid esters of glycerol, waxes, which are fatty acid esters of long-chain alcohols, and steroids such as cholesterol and ergosterol, which are derived from partially or completely derived pheanthrene. Complex lipids include, for example, phosphatides or phospholipids, which are lipids that contain phosphorous, glycolipids, which are lipids that contain carbohydrate residues, and sphingolipids, which are lipids containing sphingosine, a long-chain alcohol. The method of the invention can be used to measure the lipid antioxidant activity of the entire lipid compartment.

The methods of the invention can also be used to measure individual components of the lipid compartment. Individual lipid components can be separated from the lipid compartment of a sample using known techniques such as density gradient ultracentifugation, which separates the major lipoprotein fraction components from the other plasma proteins. Under controlled conditions, plasma would be subjected to density gradient ultracentrifugation using a vertical rotor. This procedure can be used to determine a lipoprotein cholesterol profile wherein the cholesterol concentrations of the separated lipoprotein fractions are measured. In addition, this procedure allows for the recovery of the lipoproteins distributed in the density gradient such that individual lipoproteins (i.e., VLDL, IDL, LDL, Lp(a), HDL) may be isolated. Other known methods for recovering individual lipoproteins, such as precipitation and electrophoresis, ultracentrifugation may also be used (National Cholesterol Education Program, Recommendations on Lipoprotein Measurement From the Working Group on Lipoprotein Measurement NIH Pub. No. 95-3044 (1995)).

In one embodiment of the invention, blood is isolated and used as a sample. In another embodiment, the blood is centrifuged to separate the plasma, and the plasma is used as a sample. In yet another embodiment, the lipid compartment of the sample is separated into fractions that contain individual lipid components e.g., LDL, VLDL and the like, and the separated fractions are used as a sample.

The sample may also be a non-biological sample such as food or other organic materials. Lipid oxidation products are present in unknown amounts in food producs which contain polyunsaturated fatty acids. Lipids can become rancid as a result of oxidation, which can be the cause of major food deterioration. The method of the invention may also be used to determine the oxidation of fatty acids in food products and cosmetic products.

II Free Radical Generators

The method of the invention uses free radical generators that can produce free radicals in the lipid compartment and aqueous compartment of the sample. It is generally accepted that the process of lipid oxidation in biological samples proceeds by way of a free radical mechanism called autoxidation, which can be described in terms of initiation, propagation, and termination processes. The process of lipid oxidation, in foods for example, may be initiated by a number of mechanisms including: (a) singlet oxygen; (b) enzymatic and non-enzymatic generation of partially reduced or free radical oxygen species (i.e., hydrogen peroxide, hydroxyl radical); (c) active oxygen iron complexes; and (d) thermal or iron-mediated homolytic cleavage of hydroperoxides. Details of these mechanisms can be found in a number of review articles, such as that by Stan Kubow (Kubow, Free Radic. Biol. Med. 12:63-81 (1992)) and E. N. Frankel (Frankel et al., J. Amer. Chem. Soc. 61:1908-1917 (1984)).

In one embodiment, the invention uses lipophilic radical generators that are lipid soluble. These lipophilic radical generators are able to produce high levels of free radicals in the lipid compartment of a sample. The lipophilic radical generators may generate free radicals at a constant rate and at a high efficiency e.g., MeO-AMVN. For example, although AMVN induces free radicals, it does so at a relatively slow rate, and thereby requiring a higher concentration of AMVN to induce and sustain lipid free radicals. However, the lipophilic free radical generator, MeO-AMVN, which has a rate constant for decomposition that is 15 times faster than AMVN can be used at lower concentrations (Example 4).

Suitable lipophilic radical initiators include, but are not limited to, organic hydroperoxide such as cumene hydroperoxide, tert-butytl-hydroperoxide or azo-radical generating compounds such as 2,2′-azobis(4-methoxy-2,4-dimethylvaleronitrile) (MeO-AMVN), 2,2′-azobis(2,4-dimethylvaleronitrile) (AMVN), azo-bis-isobutylnitrile, 2,2′-azobis(2-methylproprionate) (DAMP), 2,2′-azobis-(2-amidinopropane), and unsymmetrical azo compounds (i.e. 2,2′-azobis(2-amidinopropane)[2-(N-stearyl)amidinopropane], 2,2′-azo[2-(2-imidiazolin-2-yl)-propane)-[2-[2-(4-n-octyl)imidazolin-2-yl]-propane]) (Culbertson et al., Free Radic. Res. 33(6): 705-718 (2001)).

The lipophilic radical generator used should be suitable for use and detection with the oxidizable lipophilic indicator. For example, MeO-AMVN was determined to be useful when using fluorescence to monitor oxidation with C11-BODIPY 581/591 as the oxidizable lipophilic indicator.

In another embodiment, the invention uses hydrophilic radical generators that are water-soluble and which initiate free radicals in the aqueous compartments of the sample. Two widely used methods used to initiate free radicals in the aqueous compartment are to incubate with a solution of copper Cu²⁺ or with a thermally labile azo-radical generator (Goss et al., Free Radic. Res. 31: 597-606 (1999)). Examples of azo-radical generators include, but are not limited to compounds such 2,2′ azobis(2-amidinopropane)dihydrochloride (AAPH), and 2,2′-azobis[2-(5-methyl-2-imidazolin-2-yl)propane]dihydrochloride. Other examples of hydrophilic free radical generators include organic hypdroperoxide.

III Oxidizable Indicators

The method of the invention uses oxidizable indicators to measure the extent of antioxidant activity in a sample. These oxidizable indicators become oxidized in the presence of free radicals. The oxidation of the indicator produces a detectable change in the indicator, for example, a color change or a fluorescence change. Fluorescent probes are available commercially, for example from Molecular Probes (Eugene, Oreg.)

In one embodiment, the oxidizable indicator is a lipophilic oxidizable indicator. The lipophilic oxidizable indicator can be lipid soluble. Examples of lipophilic oxidizable indicators include, but are not limited to, pyrene fatty acid derivatives, perlene fatty acids, cis-parinaric acid, diphenyl-1-pyrenylphosphine (DPPP), BODIPY fatty acids (i.e., 4,4-difluoro-5-(4-phenyl-1,3-butadienyl)-4-bora-3a,4a-diaza-s-indacene-3-undecanoic acid (BODIPY 581/591 C11), (E,E)-3,5-bis-(4-phenyl-1,3-butadienyl)-4,4-difluoro-4-bora-3a,4a-diaza-s-indacene (BODIPY 665/676), hexadecanamide, N-(3′,6′-dihydroxy-3-oxospiro(isobenzofuran-1(3H),9′-(9H)xanthen)-5-yl), lipophilic fluorescein dyes (hexadecanoylaminofluorescein and fluorescein-labeled phosphatidylethanolamine).

Fluorescent fatty acid analogs, such as BODIPY fatty acids (Naguid, U.S. Pat. No. 6,060,324 (2000); Naguid U.S. Pat. No. 6,114,177 (2000)), have been used as oxidizable lipophilic indicators to detect peroxyl radicals in organic solvent mixtures, or liposome suspensions, and can be used in the present invention. Their lack of ionic charge, which allows exclusive localization to the lipid compartment, together with their oxidation sensitive conjugated double bonds, and long-wavelength fluorescence make BODIPY fatty acids suitable oxidizable lipophilic indicators. Upon interacting with peroxyl radicals, the BODIPY oxidizable indicator produces a detectable change in fluorescence. However, if antioxidants intercept the free radicals, BODIPY will retain its original florescent signal. Therefore, this method can be used as an indirect assay of the radical scavenging ability of antioxidants. The lipophilic oxidizable indicator is added to the sample preferably at approximately the same time as the lipophilic radical generator. Experiments detailed in the Examples section below show the use of a fluorescent lipophilic oxidizable indicator BODIPY 581/591 C11, which is a suitable lipophilic oxidizable indicator that can be used to determine the antioxidant activity of the lipid compartment of a biological sample.

The use of fluorescent oxidizable indicators and fluorometric measurements of the indicator is one method of directly monitoring the antioxidation activity of the sample. Pyrene fatty acid derivatives are also useful as oxidizable indicators because they are susceptible to oxygen quenching due to their long excited-state lifetimes, and therefore can be efficiently used to measure oxygen concentration. cis-Parinaric acid is a natural polyunsaturated fatty acid that is structurally similar to membrane lipids. Spectroscopically, cis-parinaric acid may also be a useful as a lipophilic oxidizable indicator to evaluate antioxidant activity since it has a large fluorescence Stokes shift (approx. 100 nm) and almost completely lacks fluorescence in aqueous solutions. The large degree of unsaturation of cis-parinaric acid makes it susceptible to oxidation by the free radical. Another suitable oxidizable lipophilic indicator may be the lipid soluble diphenyl-1-pyrenylphosphine (DPPP), which is non-fluorescent until oxidized to a phosphine oxide by peroxides and thus may be used to monitor the production of hydroperoxides in lipids. Lipophilic fluorescein dyes, such as headecanoylaminofluorescein, and fluorescein labeled phosphatidylethanolamine, may also be employed to monitor peroxyl radical formation in the method of the invention.

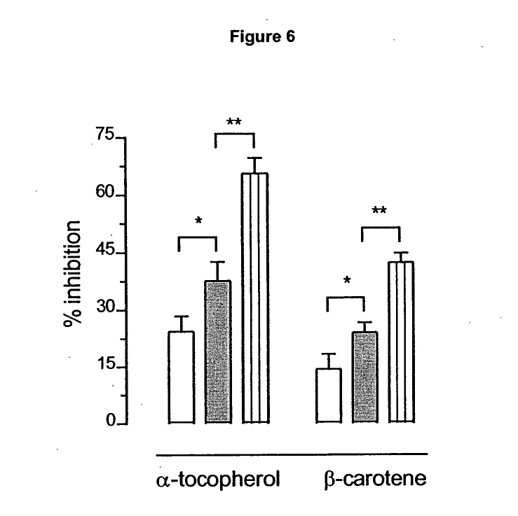

To validate the analysis of lipid oxidizability, a study was done to determine the effect of the lipid soluble antioxidants, α-tocopherol and β-carotene on the oxidation, or the antioxidant activity of lipid compartment of plasma. α-Tocopherol or β-carotene was added into the plasma before incubating with the lipophilic radical generator, MeO-AMVN as described in Example 1. Results from Example 5 and FIG. 6 show that antioxidants were effective in protecting the oxidation of lipophilic probe, BODIPY. The protective effect was significantly increased depending on the duration of pre-incubation, 1 & 6 hr, of the antioxidants. These data suggest that lipid soluble antioxidants, such as α-tocopherol and β-carotene can be incorporated in the lipophilic compartment by incubating with BODIPY, and that the BODIPY is localized in the lipophilic compartment of plasma.

In addition, the examples also shows that BODIPY is highly lipophilic and almost exclusively localizes in the lipid compartment. Density gradient ultracentrifugation of the lipid compartment allows for the separation of lipoprotein fractions (i.e., VLDL, IDL, LDL, Lp(a), and HDL) within plasma. A plasma sample following the addition of BODIPY was subjected to ultracentrifugation and then monitored for fluorescence. Only the bands correlating to the lipoprotein fractions yielded red fluorescence, associated with BODIPY, indicating that BODIPY was very specific for the lipid compartment. Accordingly, the method of the invention can be used to determine the antioxidant activity of separate lipoprotein fractions. This can be useful for diagnostic information regarding diseases associated with oxidative stress of specific lipoproteins. Furthermore, the results demonstrated that BODIPY was able to localize within each fraction of the plasma producing a red fluorescence associated with each band corresponding to VLDL, IDL, LDL, Lp(a) and HDL.

In another embodiment, the oxidizable indicator is a hydrophilic oxidizable indictor. Examples of hydrophilic oxidizable indictors include, but are not limited to, DCFH (2′,7′-dichlorodihydrofluorescein); 4,4-difluoro-5,7-dimethyl-4-bora-3a,4a-diaza-s-indacene-3-propionyl ethylenediamine, hydrochloride, and R-phycoerythrin.

IV Antioxidants Compositions

In one aspect, the method of the invention relates to providing protection against free-radical induced disorders by administering antioxidants. Antioxidants can be characterized in different ways based upon their solubility, their mechanism, or their localization site within the body. Antioxidants can either be fat soluble (lipophilic), water soluble (hydrophilic) or both (Halliwell et al. Arch. Biochem. Biophys. 280:1-8 (1990)). Lipophilic antioxidants, such as carotenoids, can protect the cell membrane and enter the cell to protect other parts of the cell that are surrounded by lipid membranes. However, since it cannot dissolve in the blood, lipophillic antioxidants are transported attached to another molecule. Hydrophilic antioxidants, such as vitamin C, act in the blood. Since they cannot dissolve in the lipid membrane, they must be specifically transported into the cell where it can protect the aqueous parts of the cell. Some antioxidants, such as alpha lipoic acid and vitamin E, are both lipophilic and hydrophilic and hence can provide protection almost anywhere in the body. Antioxidants also differ in the class of free radicals (e.g. hydroxyl anion or singlet oxygen) that they can neutralize. For example, vitamin E is effective against peroxyl radicals, singlet oxygen, and peroxynitrite whereas carotenoids only protect against singlet oxygen or peroxyl radicals. Additionally, antioxidants can act as primary antioxidants, which decrease the initiation rate of peroxidation (i.e. transferrin and ceruloplasmin bind prooxidant metal ions) or as secondary antioxidants, which decrease the chain propagation and amplification of peroxidation (i.e. α-tocopherol scavenges oxidizing species). However, most antioxidants are not exclusive, but act with multiple antioxidant properties (e.g., uric acid) (Halliwell et al., Arch. Biochem. Biophys. 280:1-8 (1990)). Therefore, an accurate determination of the total antioxidant activity requires assessment of the net effect of all antioxidants present in a sample rather than individually analyzed antioxidants. The present invention provides a method for determining the net antioxidant effect of all classes of antioxidants using the oxidizable lipophilic and hydrophilic oxidizable indicators.

Antioxidants also accumulate in and protect different parts of the body. For example, vitamin C accumulates in the lens of the eye providing protection from cataracts. The carotenoids β-carotene and lutein accumulate in the skin and protect it from the sun's damaging rays. Lutein also accumulates in the macula of the eye, reducing oxidative stress and the risk of macular degeneration. Vitamin E is absorbed into cell membranes, protecting them from oxidative stress. Coenzyme Q10 protects mitochondria from free-radical damage. Some bioflavonoids are thought to be important in protecting the integrity of blood vessels.

The method of the invention can be used to provide protection in the all the compartments of the sample, i.e., in both the aqueous compartment and the lipid compartment. In another embodiment, the method of the invention relates to providing protection in a particular compartment, e.g., the lipid compartments or the aqueous compartment. The protective effects of antioxidants, α-tocopherol and β-carotene pre-incubated with plasma, are shown in Example 5. The protective effects of antioxidant, EGCG are shown in Example 6.

The methods of the present invention can be used to maintain, or administer proper levels of physiologically acceptable antioxidants in an individual. For example, an individual under undergoing a cholesterol-lowering regimen often has reduced serum levels of biological antioxidants such as β-carotene, vitamin A, vitamin E and vitamin C. Although the mechanism of this action is unclear, the lowering of antioxidants may be due to the fact that β-carotene and vitamins A and E are fat- or lipid-soluble. Thus, as the individual's lipid levels decrease through use of a cholesterol-lowering agent, less lipid is available to solubilize the antioxidants and less antioxidant is available to the body. Individuals having reduced levels of serum antioxidants as a result of a cholesterol-lowering agent may have an increased risk of developing cancer. (See e.g., Stahelin et al., Am J Epidemiology. 133:766-775 (1991)).

Lipid soluble antioxidants include, but are not limited to, carotenoids such as lutein, zeaxanthin, β-cryptoxanthin, trans-lycopene, total lycopene, α-carotene, trans-β-carotene, total-β-carotene; tocopherols (vitamin E) such as α-tocopherol, gamma-tocopherol and delta-tocopherol; retinoids (vitamin A) such as retinol, retinyl palmitate and Ubiquinone—Coenzyme Q10.

Examples of aqueous antioxidants include, but are not limited to, ascorbic acid and its oxidized form, “dehydroascorbic acid”, uric acid and its oxidized form, “allantoin,” bilirubin, albumin, vitamin C, and water-soluble polyphenols such as catechins, isoflavones, and procyanidins.

Compositions within the scope of the invention comprise at least one physiologically acceptable antioxidant. For example, several vitamins may act as biological antioxidants including β-carotene, vitamin A, vitamin C and vitamin E. These vitamins appear to work at different levels of carcinogenesis. (Stahelin et al., Am J Epidemiology 133:766-775 (1991)). B-carotene may act as a scavenger for free radicals in the body. Vitamin A (retinol) has been recognized as being able to interfere with carcinogenesis. (See Goodman Gilman, The Pharnacological Basis of Therapeutics, Pergamon Press, New York (1990)). It is likely that vitamin A acts at the promotion or progression phase of carcinogenesis. Vitamin C (ascorbic acid) may also act as an antioxidant by preventing nitrosamine formation in the stomach and reducing fecal mutagenicity. Vitamin E (α-tocopherol), when acting as an antioxidant, may inhibit the formation of carcinogenic promoters by protecting essential cellular constituents, such as the polyunsaturated fatty acids of cell membranes, from peroxidation and by preventing the formation of toxic oxidation products. These and other physiologically acceptable antioxidants are within the scope of the invention. Also within the scope of the invention are combinations of antioxidants, such as combinations of aqueous antioxidants, lipid antioxidants, or combinations with both aqueous and lipid antioxidants.

The dosage range for other physiologically acceptable antioxidants is determined by reference to the usual dose and manner of administration of the antioxidant. For example, a range of from about 15 mg to about 1000 mg/day of vitamin E; from about 50 mg to about 2000 mg/day of vitamin C; from about 900 μg to about 3000 μg/day of vitamin A, from about 50 μg to 400 μg/day of selenium, and from 5 to 30 μg/day of carotenoid. The composition or combination of agents should be administered in amounts sufficient to ensure that the serum level of antioxidants is maintained at an appropriate level or restored or increased to an appropriate level while serum cholesterol levels are reduced.

One or more physiologically acceptable antioxidants composition can be formulated in form suitable for topical application. For example, as a lotion, aqueous or aqueous-alcoholic gels, vesicle dispersions or as simple or complex emulsions (O/W, W/O, O/W/O or W/O/W emulsions), liquid, semi-liquid or solid consistency, such as milks, creams, gels, cream-gels, pastes and sticks, and can optionally be packaged as an aerosol and can be in the form of mousses or sprays. The composition can also be in a sunscreen. These compositions are prepared according to the usual methods. The composition can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507.

One or more physiologically acceptable antioxidants can be administered as compositions by various known methods, such as by injection (subcutaneous, intravenous, etc.), oral administration, inhalation, transdermal application, or rectal administration. Depending on the route of administration, the composition containing the antioxidant may be coated with a material to protect the compound from the action of acids and other natural conditions which may inactivate the antioxidant. The composition can further include both the antioxidant an a cholesterol-lowering agent.

To administer the composition by other than parenteral administration, it may be necessary to coat the composition with, or co-administer the composition with, a material to prevent its inactivation. For example, the composition may be administered to a subject in an appropriate diluent or in an appropriate carrier such as liposomes. Pharmaceutically acceptable diluents include saline and aqueous buffer solutions. Liposomes include water-in-oil-in-water CGF emulsions as well as conventional liposomes (Strejan et al., J. Neuroimmunol. 7:27 (1984)).

The composition containing at least one antioxidant may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as licithin, by the maintenance of the required particle size in the case of dispersion and by the use of surfactants. Prevention of the action of microorganisms can be achieved by various antibacterial and antifungal agents. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition containing the antioxidant in the required amount in an appropriate solvent with one or a combination of ingredients enumerated above, as required. Generally, dispersions are prepared by incorporating the composition into a sterile vehicle which contains a basic dispersion medium and the required other ingredients from those enumerated above.

When the composition containing the antioxidant is suitably protected, as described above, the composition may be orally administered, for example, with an inert diluent or an assimilable edible carrier. The composition and other ingredients may also be enclosed in a hard or soft shell gelatin capsule, compressed into tablets, or incorporated directly into the subject's diet. For oral therapeutic administration, the composition may be incorporated with excipients and used in the form of ingestible tablets, buccal tablets, troches, capsules, elixirs, suspensions, syrups, wafers, and the like. The percentage of the compositions and preparations may, of course, be varied. The amount of active compound in such therapeutically useful compositions is such that a suitable dosage will be obtained.

The tablets, troches, pills, capsules and the like may also contain a binder, an excipient, a lubricant, or a sweetening agent. Various other materials may be present as coatings or to otherwise modify the physical form of the dosage unit. For instance, tablets, pills, or capsules may be coated with shellac, sugar or both. Of course, any material used in preparing any dosage unit form should be pharmaceutically pure and substantially non-toxic in the amounts employed. As used herein “pharmaceutically acceptable carrier” includes any solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in compositions of the invention is contemplated.

It is especially advantageous to formulate compositions of the invention in dosage unit form for ease of administration and uniformity of dosage. Dosage unit form as used herein refers to physically discrete units suited as unitary dosages for the subjects to be treated. Each dosage contains a predetermined quantity of active compound calculated to produce the desired therapeutic effect in association with the required pharmaceutical carrier. The specification for the novel dosage unit forms of the invention is dependent on the unique characteristics of the composition containing the antioxidant and the particular therapeutic effect to be achieved. Dosages are determined by reference to the usual dose and manner of administration of the ingredients.

V Uses

Many disorders or diseases arise due to oxidative stress and the presence of free radicals. The method of the invention can be used to help diagnose, monitor, and assess treatment of disorders associated with antioxidant levels and excess free radicals. The method is accurate, quick, non-invasive, and can be easily adapted for high throughput usage and diagnostic procedures. Large populations of individual can be screened for people afflicted with a certain disease state for deviations in antioxidant levels may allow new correlations between disease and antioxidant levels to be found. For example, aging at a higher than normal rate, segmental progeria disorders, Down's syndrome; heart and cardiovascular diseases such as atherosclerosis, adriamycin cardiotoxicity, alcohol cardiomyopathy; gastrointestinal tract disorders such as inflammatory & immune injury, diabetes, pancreatitis, halogenated hydrocarbon liver injury; eye disorders such as cataractogenesis, degenerative retinal damage, macular degeneration; kidney disorders such as autoimmune nephrotic syndromes and heavy metal nephrotoxicity; skin disorders such as solar radiation, thermal injury, porphyria: nervous system disorders such as hyperbaric oxygen, Parkinson's disease, neuronal ceroid lipofuscinoses, Alzheimer's disease, muscular dystrophy and multiple sclerosis; lung disorders such as lung cancer, oxidant pollutants (O₃,NO₂), emphysema, bronchopulmonary dysphasia, asbestos carcinogenicity; red blood cell disorder such as malaria Sickle cell anemia, Fanconi's anemia and hemolytic anemia of prematurity; iron overload disorders such as idiopathic hemochromatosis, dietary iron overload and thalassemia; inflammatory-immune injury, for example, glomerulonephritis, autoimmune diseases, rheumatoid arthritis; ischemia reflow states disorders such as stroke and myocardial infarction; liver disorder such as alcohol-induced pathology and alcohol-induced iron overload injury; and other oxidative stress disorders such as AIDS, radiation-induced injuries (accidental and radiotherapy), general low-grade inflammatory disorders, organ transplantation, inflamed rheumatoid joints and arrhythmias. The method of the invention can be used for diagnosis and prevention of a free radical induced disorder, or an oxidative stress disorder.

(i) Diagnostic Screening Assay

The methods of the invention can be used to provide lipid profile for a subject by determining the antioxidant activity of the lipid fractions of the lipid component and profile for lipoproteins such as cholesterol, HDL cholesterol, LDL cholesterol, apolipoprotein B, apolipoprotein A1, triglycerides, LDL/HDL ratio and LDL/ApoB ratio. Based on this profile, the appropriate course of one or more antioxidants may be administered. For example, with ischemic heart disease, the level of LDL is low and there is a reduced antioxidant activity in this fraction. Accordingly, a lipid soluble antioxidant may be administered to raise the antioxidant activity in the LDL fraction of the subject. For example, LDL is a main carrier of non-polar carotenoids such as β-carotene and lycopene, LDL and HDL transport polar carotenoid such as lutein and zeaxanthin).

In one embodiment, an individual's antioxidant activity can be compared with a population average. It is reasonable to predict that the lower the antioxidant level, the higher the likelihood that health problems will develop. In another embodiment, the subject's antioxidant activity can be compared with the average from a sub-population of individuals, for example, those of a particular group in which a pattern of antioxidant activity is associated with a higher propensity for a disorder.

The correlation of antioxidant status with disease development can further be used to identify ranges of antioxidant status which signify a risk factor, e.g., a risk of development of a particular disease. One correlation of particular relevance is the association of a lower lipid or total plasma antioxidant activity range with a predisposition indicates predisposition of an individual to the occurrence or recurrence of heart disease.

(ii) Prevention

The method of the invention can also be used in conjunction with other medical data, where a physician can advise patients whether they are at unusual risk for a free radical associated disorder and what action to take to prevent the disorder or delay its onset. For example, the addition of specific antioxidants to the diet may help reduce the individual's risk to disease.

(iii) Treatment

The method of the invention can also be used to monitor the antioxidant status of an individual suffering from a free radical disorder. The antioxidant status of the individual can be altered by therapeutic treatment with an antioxidant regimen. The method of the invention can also be used to provide useful information for the ongoing treatment of the individual.

(iv) Food Agricultural Use

The methods of the invention can also be used as a quality control for food manufacturing and processing. Food products represent an important source of essential antioxidants. However, different strains of vegetables, fruits, or any other plant can have wide differences in antioxidant content depending on breeding, cultivation, harvesting and processing conditions. Quality control during food manufacturing and processing can benefit from close monitoring of antioxidant status. The method of the invention can be used to assess the antioxidant content of plants as well as food products to help determine food processing conditions.

(v) Cosmetics

Sagging skin and other signs of degenerative skin conditions, such as wrinkles and age spots are caused primarily by free radical damage. Vitamin C has been shown to accelerate wound healing, protect fatty tissues from oxidation damage, as well as play an integral role in collagen synthesis (Zhang et al., Bioelectrochem Bioenerg 48:453-61 (1999)). Clinical studies show that antioxidants in a cosmetic vehicle can inhibit the induction of lipid peroxidation in stratum corneum lipids, which are produced endogenously or induced by UVB exposure (Pelle et al., Photodermatol Photoimmunol Photomed 15:115-119 (1999)).

α-Tocopherol has been shown to be the major antioxidant in the human stratum corneum. Depletion of α-tocopherol is an early and sensitive biomarker of environmentally induced oxidation. Topical and/or systemic application of antioxidants could support physiological mechanisms that maintain or restore a healthy skin barrier and protect the skin from environmental stresses that may lead to UV-induced carcinogenesis, photoaging, or desquamatory skin disorders (Thiele et al., Curr Probl Dermatol 29:2642 (2001)).

The method of the invention can be used in monitoring the effectiveness of new topical cosmetic products as well as in studying the protective mechanism of antioxidants. In addition, the method of the invention could be used to monitor levels of antioxidants, in particular, α-tocopherol, a biomarker for environmentally induced oxidation, in order to assess a subject's level of environmentally-caused skin damage or aging.

This invention is further illustrated by the following examples which should not be construed as limiting. The contents of all references, patents and published patent applications cited throughout this application, are incorporated herein by reference.

### EXAMPLES

The following experiments were performed to establish a selective fluorescent method to measure oxidation of the aqueous and lipid compartments of a biological sample. In particular, a lipid-soluble radical initiator, MeO-AMVN, together with a lipid fluorescence probe, BODIPY 581/591-C11, were used to study the plasma lipid oxidizability.

### Example 1

### Materials and Methods

(i) Chemicals

The radical initiators AAPH, AMVN and MeO-AMVN were obtained from Wako Chemicals (Richmond, Va., USA). The fatty acid analogue C11-BODIPY 581/591 and 2′,7′-dichlorodihydrofluorescein diacetate (DCFH-DA) were obtained from Molecular Probes (Eugene, Oreg., USA). (−)-Epigallocatechin-(3)-gallate (EGCG), α-tocopherol were purchased from Sigma (St. Louis, Mo., USA). 2,2-diphenyl-1-picrylhydrazyl (DPPH) was from Fluka (Milwake, Wis.). All-trans-β-Carotene (type II), α-tocopherol, lycopene, and bovine serum albumin (BSA) were purchased from Sigma Chemical Co. (St Louis, Mo., USA). Lutein was purchased from Kemin Industries (Des Moines, Iowa, USA). Zeaxanthin, cryptoxanthin and echinenone were gifts from Hoffinann-La Roche (Nutley, N.J., USA). All other reagents were of analytical grade.

(ii) Human Plasma Oxidation Induced by Water- and Lipid-Soluble Radical Inducers

After an overnight fast (10-12 h), blood from two healthy donors (32 and 35 years old) was collected in ethylenediaminetetraacetic acid (EDTA)-containing tubes. In order to reduce the variability of different donors, blood samples from these two subjects were collected weekly for the duration of the experiment. Immediately after collection, the samples were placed on ice and protected from light. Plasma was obtained by centrifugation at 800 g for 20 min at 4° C. and immediately used for the in vitro studies.

Aqueous and lipid plasma oxidation was induced at a constant rate by the two azo-initiators: 1) AAPH as a water-soluble peroxyl radical generating system, 2) AMVN and the analogue MeO-AMVN as lipid-soluble peroxyl radical initiators.

In order to compare the consumption of endogenous antioxidants induced by AAPH and MeO-AMVN, the amount of free radicals generated was kept constant by adjusting the concentration of the two azo-initiators. In the presence of 10-20 mM of AAPH, the flux of aqueous radicals calculated on the basis of the known rate of free radical generation from AAPH at 37° C. (Rᵢ=1.36×10⁻⁶ [AAPH] mol/liter/sec) (Niki, Methods in Enzymology. Oxygen radicals in biological systems 186: 100-108 (1990)) was respectively 1.36 and 2.72×10⁻⁸ mol/liter/sec. Since the rate of peroxyl radical formation from MeO-AMVN was 14.2×10⁻⁶ mol/liter/sec (calculated in micelles) (Noguchi et al., Free Rad. Biol. Med. 24:259-268 (1998)) the concentration of the lipophilic azo-initiator was reduced 10 fold to 1-2 mM), to reach the same order of free radical flux.

AAPH was prepared in phosphate buffered saline (50 mM, pH 7.4, PBS) and stored at −20° C., while AMVN and MeO-AMVN were prepared respectively in EtOH and CH₃CN immediately before use. In order to obtain homogeneous incorporation, the lipid soluble initiators were added slowly to the samples with a micro-syringe (10 μl) with stirring. The samples were then vortexed for 10 sec and incubated at 37±1° C. under aerobic conditions.

(iii) Determination of Hydrophilic and Lipophilic Plasma Antioxidants

Since the fluorescence probes did not affect the plasma concentration of antioxidant nutrients (data are not shown), the probes were not added in the incubation for the antioxidant nutrient analysis. Plasma:PBS (1:5, by vol) was incubated at 37° C. up to 4 hr in the presence and absence of the hydrophilic radical generator, AAPH (10 mM and 20 mM) or the hydrophobic radical generator, MeO-AMVN (1 mM and 2 mM).

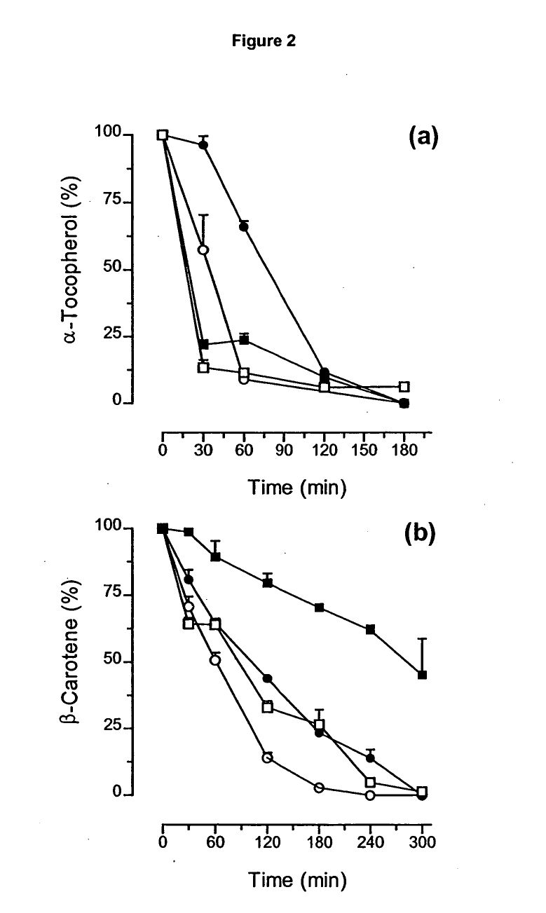

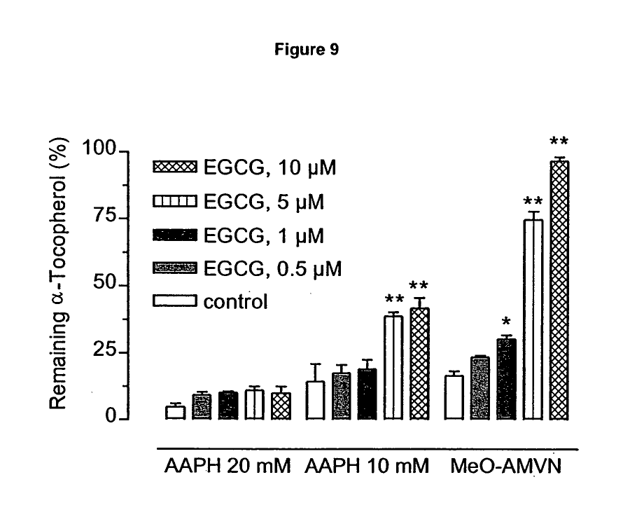

In the first experiment, the fat-soluble antioxidant nutrients, such as β-carotene and α-tocopherol, were measured at 30 min, 1 hr, 2 hr, 3 hr and 4 hr. β-Carotene and α-tocopherol in plasma were extracted and measured using the HPLC method described earlier (Yeum et al., Am. J. Clin. Nutr. 64:594-602 (1996)). The results of this experiments are shown in FIG. 2. In a second similar experiment, other antioxidants were studied. After 60 min of incubation in aerobic conditions, the fat-soluble antioxidant nutrients, such as α-tocopherol, β-carotene, lycopene, cryptoxanthine, zeaxanthine and lutein were extracted and measured using the HPLC method described earlier (Yeum et al. Supra), the results of which are shown in FIG. 9. A 100 μl aliquot of the reaction mixture was extracted for β-carotene and α-tocopherol analysis. Echinenone in ethanol was added as an internal standard. The mixture was extracted with CHCl₃:CH₃OH (2:1 v/v) containing 0.2% BHT and hexane containing 0.1% BHT, dried under nitrogen, redissolved in ethanol, and injected into an HPLC system with a C30 column (3 μm, 150×4.6 mm, YMC, Wilmington, N.C.). A Waters 994 programmable photodiode array detector was set at 450 nm for carotenoids and 292 nm for α-tocopherol analyses.

The major water-soluble antioxidants (ascorbic acid and uric acid) were measured at 5 min, 15 min, 30 min, 1 hr, 2 hr, 3 hr and 4 hr. For water-soluble antioxidant measurement, the mixtures were immediately deproteinized with perchloric acid (250 mM). Ascorbic acid and uric acid in plasma was analyzed by HPLC using an electrochemical detector (Bioanalytical System, Inc, N. Lafayette, Ind.) as described earlier (Behrens et al., Anal. Biochem. 165:102-107 (1987)).

Results are expressed as percentages with respect to control samples prepared without the azo-compounds.

(iv) Measurement of Plasma Oxidation

Plasma oxidation was measured fluorometrically using two different fluorescent probes: DCFH and BODIPY. DCFH-DA and BODIPY stock solutions were prepared in EtOH and dimethylsulfoxide, respectively, stored under nitrogen at −20° C. and used within two months. The final plasma dilution was 1:5 (v/v).

DCFH was prepared from DCFH-DA by basic hydrolysis. Briefly 500 μl of DCFH-DA stock solution (1 mM) was mixed with 2 ml of NaOH (0.01 N at 4° C.) for 20 minutes while protected from the light. The mixture was then neutralized with 2 ml of HCl (0.01 N), diluted with PBS to a final concentration of 10 μM and stored in ice for no longer than 8 hrs (working solution); an aliquot of 100 μl was added to 200 μl of plasma and then diluted to a final volume of 1 ml with PBS. Aqueous plasma oxidation was measured monitoring the 2-electron oxidation of DCFH to the highly fluorescent compound 2′,7′-dichlorofluorescein (DCF). The excitation wavelength (λex) was set at 502 nm (slit 5 nm) and emission (λem) at 520 nm (slit 5 nm).

For BODIPY incorporation into the lipid plasma compartment, 25 μl of the BODIPY stock solution (2 mM) were diluted 100-fold with PBS. Aliquots of 100 μl were then added to 200 μl of plasma and 100 μl of PBS, vortexed for 20 sec and then incubated under aerobic conditions for 10 minutes at 37° C. The final volume was adjusted to 1 ml with PBS yielding BODIPY at a final concentration of 2 μM. Lipid plasma oxidation was determined by monitoring both the red fluorescence decay (λex=580, λem=600 nm) of BODIPY and the green fluorescence increase (λex=500, λem=520 nm) of the oxidation product. In the experiments using β-carotene, to avoid the filtering effect due to the carotenoid, the oxidation product of BODIPY was also detected at λex=520 and at λem=540 nm. The fluorescence measurements were carried out using a Perkin Elmer spectrofluorometer (model 650-10s) with 1 cm path length fluorescence cuvettes.

In order to evaluate the intra-assay precision of the method, six replicates of the same plasma sample were analyzed by a single individual while the inter-assay repeatability was carried out by four different individuals. The precision was evaluated as coefficient of variation (CV).

In order to evaluate the consumption of the antioxidant, (−)-epigallocatechin-(3)-gallate (EGCG) (See Example 7), the EGCG was prepared in cold PBS immediately before the usage and added to plasma samples at a final concentration of 0.5, 1, 5 and 10 μM.

(v) α-Tocopherol and β-Carotene Plasma Enrichment

Plasma was supplemented with α-tocopherol and β-carotene according to Bowen and Omaye (Bowen, et al. J. Am. Coll. Nutr. 17:171-179; 1998) with minor modifications. Briefly, β-carotene (dissolved in stabilized THF, 10 mg/ml) or α-tocopherol (4.3 mg/ml in EtOH) were added to plasma to reach a final concentration of 50 μM; the samples were then vortexed for 30 sec and incubated at 37±1° C. for 1 to 6 hr under nitrogen. After the pre-incubation period, plasma samples were diluted 5-fold with PBS to give a final concentration of α-tocopherol and β-carotene of 10 μM; the final amount of the solvents was always less than 0.8% v/v. Controls were prepared in the same way using solvent only.

(vi) ESR Experiments

Tocopheroxyl radicals (TOC-O.) were generated by reaction of α-tocopherol and DPPH according to Eq. (1) as described by Rousseau-Richard (Rousseau et al., FEBS Lett. 233(2):307-10 (1988)).

α-TOC+DPPH→α-TOC-O.+DPPHH [1]

For sample preparation, α-tocopherol (900 μM) and DPPH (600 μM) in ethanol solution were mixed for 20 sec and 50 μl of the reaction mixture transferred into a capillary ESR tube. (−)-Epigallocatechin-(3)-gallate (EGCG) was added to the mixture immediately after DPPH decolorization (30 sec after DPPH addition). After exactly 60 sec from the starting of the reaction, the ESR spectra were recorded at room temperature with a Bruker EMX spectrometer at 9.5 GHz (X band) equipped with a cylindrical cavity (ER4119HS; Bruker) and in the following instrumental conditions: microwave frequency, 9.316 GHz; microwave power, 15 mW; modulation 2 G; number of scans, 1; resolution, 1024 points. The spectra were recorded and doubly integrated by using a Bruker WINEPR system (version 2.11).

(vii) Statistical Analysis

Results were expressed as mean±SEM. Statistical analysis were performed with a one-way of analysis (ANOVA) followed by Dunnett's post-test. GraphPad Prism (version 2.01) (GraphPad Software, Inc) was used for all analyses. A p value less than or equal to 0.05 was considered significant.

### Example 2

### Plasma Antioxidant Consumption Induced by AAPH and MeO-AMVN

To determine plasma antioxidant consumption induced by lipophilic and hydrophilic radical generators, plasma was incubated in the presence of AAPH (hydrophilic generator) and MeO-AMVN (lipophilic generator) as described in Example 1(ii), and the oxidation measured as described in Example 1(iii) and (vi).

The results of the study show that the major hydrophilic (ascorbic acid and uric acid) and lipophilic (α-tocopherol and β-carotene) plasma antioxidants were consumed in a time-dependent manner in the presence of AAPH or MeO-AMVN. As expected by the solubility of the radical inducers, the hydrophilic antioxidants were consumed more rapidly when AAPH was used, in contrast to MeO-AMVN.

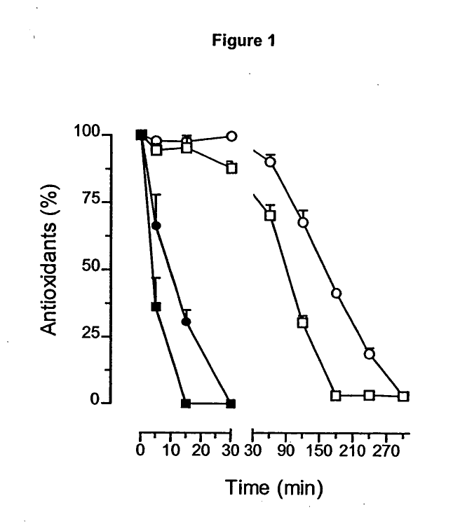

FIG. 1 shows the effect of AAPH and MeO-AMVN on hydrophilic antioxidants levels in human plasma. The symbols in FIG. 1 are: AAPH (20 mM): AA (▪), UA (□); MeO-AMVN (2 mM): AA (●), UA (∘). Values are mean±SD of three independent experiments. The initial concentrations of ascorbic acid (AA) and uric acid (UA) were respectively 48 μM and 220 μM. The azo-compounds were added to plasma samples (1:5 with PBS) and incubated at 37° C. in the dark. At fixed times, aliquots were withdrawn and the concentration of AA and UA assayed by HPLC as described in the text. The results from FIG. 1 show that ascorbic acid and uric acid were completely consumed within 15 min and 180 min, respectively using 20 mM AAPH. The consumption of these antioxidants was significantly slower in the presence of 2 mM MeO-AMVN since total disappearance of ascorbic acid and uric acid was observed after 30 min and 300 min, respectively.

FIG. 2 shows the effect of AAPH and MeO-AMVN on α-tocopherol (A) and β-carotene (B) levels in human plasma (1:5 with PBS). The symbols in FIG. 2 are: AAPH 10 mM (▪), AAPH 20 mM (□), MeO-AMVN 1 mM (●), MeO-AMVN 2 mM (∘). Values are mean±SD of three independent experiments. The initial concentration of the lipophilic antioxidants was 25 μM (α-tocopherol) and 3 μM (β-carotene). In the presence of 10-20 mM AAPH, the lipophilic antioxidant α-tocopherol was almost completely consumed within 30 min (FIG. 2A), whereas there was little oxidation of β-carotene in this period (FIG. 2B). In the presence of 2 mM MeO-AMVN, the α-tocopherol content was reduced by 42% at 30 min, and almost totally depleted after 60 min of incubation. The rate of consumption was significantly lower at 1 mM MeO-AMVN. In contrast to the consumption of ascorbic acid, uric acid and α-tocopherol, the kinetics of β-carotene depletion was faster in the presence of 2 mM MeO-AMVN as compared to that of 10-20 mM AAPH (FIG. 2B).

The distribution in aqueous and lipid compartments of the two radical initiators was determined by measuring the rate of consumption of the plasma hydrophilic and lipophilic endogenous antioxidants in the plasma.

In the presence of AAPH (20 mM), the following order of disappearance of antioxidants was observed: ascorbic acid>α-tocopherol>uric acid and β-carotene indicating a gradient of peroxyl radicals from the aqueous to the lipid phase. Ascorbic acid could effectively trap hydrophilic peroxyl radicals in the aqueous phase of plasma before they are able to diffuse into the lipid phase (Frei, In: Packer, L.; Fuchs, J., eds. Vitamin E in health and disease. New York: Marcel Dekker Inc.; 1993:131-139). Similar consumptions of uric acid and β-carotene indicate that once ascorbic acid has been completely consumed, the remaining water-soluble antioxidants provide only a partial trap for the aqueous peroxyl radicals, which are then free to diffuse into the lipoproteins.

When MeO-AMVN (2mM), was used as the radical inducer, the order of disappearance was partially reversed with α-tocopherol≅ascorbic acid>β-carotene>>uric acid. β-carotene was consumed earlier than uric acid and almost at the same time as α-tocopherol, reflecting the diffusion and activation of MeO-AMVN in the lipophilic phase. The consumption of ascorbic acid by the lipophilic radical inducer, MeO-AMVN, suggests that ascorbic acid can repair the α-tocopheroxyl radical thereby regenerating α-tocopherol, and permitting it to function again as a free radical chain-breaking antioxidant (May, FASEB J. 13:995-1006 (1999), and Buettner, Arch Biochem Biophys. 300:535-543 (1993). α-tocopherol appears to be unable to trap the MeO-AMVN-derived lipid peroxyl radicals efficiently enough to prevent them from either attacking plasma lipids or from diffusing into the aqueous compartment. The consumption of uric acid by MeO-AMVN indicates that consumption of the fat-soluble antioxidants (e.g., α-tocopherol and β-carotene) probably resulted in movement of lipid radicals from lipid compartment to aqueous compartment. The rate of BODIPY oxidation (increase in green fluorescence) significantly increased after the depletion of endogenous α-tocopherol and β-carotene, whereas it was delayed for 180 min when AAPH was used instead of MeO-AMVN.

The oxidation of α-tocopherol at a more rapid rate by AAPH than by MeO-AMVN can be explained by considering the orientation of α-tocopherol in the lipid compartment. The chroman head group of tocopherol is oriented toward the membrane interfacial region whereas the phytyl side chain is embedded within the hydrocarbon region of lipid compartment. Since the head group is responsible for scavenging radicals, it would be expected to react more rapidly with the aqueous radicals generated from AAPH than with the radicals produced by MeO-AMVN, as the latter diffuses into the core of the lipoproteins.

### Example 3

### Measurement of Plasma Aqueous Compartment Oxidation

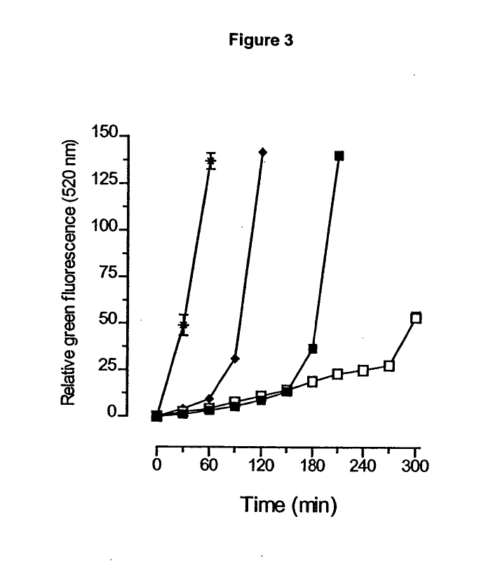

To measure the oxidation of the plasma aqueous compartment the following experiments were performed using the hydrophilic radical initiator, AAPH and the lipophilic initiator MEO-AMVN were used to generate radicals in the plasma as described in Example 1, and the oxidation of the plasma was detected. FIG. 3 shows the oxidation of DCFH to DCF induced by AAPH or MeO-AMVN. The symbols in FIG. 3 are: [symbol] (AAPH 20 mM; no plasma addition), ♦ (AAPH, 10 mM), ▪ (AAPH 20 mM), □ (MeO-AMVN, 2 mM). Values are mean±SD of five independent experiments. The reaction mixture consisted of DCFH (1 μM final concentration), the azo-compound and human plasma (1:5 with PBS). Samples were incubated at 37° C. in the dark and at fixed times the DCF content measured by fluorescence (λex=502 nm, λem=520 nm).

In the absence of plasma, 20 mM AAPH rapidly oxidized a solution of DCFH in PBS as shown in FIG. 3, where a rapid increase of fluorescence was observed which increased linearly with time. In the presence of plasma, a lag time was observed whose length was dependent on the amount of AAPH added. The propagation phase started at 90 min with 20 mM AAPH and at 180 min with 10 mM AAPH, corresponding to the depletion of both ascorbic acid and uric acid (FIG. 1). MeO-AMVN (2 mM) induced the propagation phase only after 270 min of incubation. No significant DCF formation was observed in the absence of the radical initiators until 5 hours of incubation (data not shown).

The results demonstrate that DCFH is a water-soluble indicator of radical-mediated oxidation. DCFH was used in the presence of AAPH to measure aqueous plasma oxidation. The selectivity of the method was confirmed inasmuch as DCFH oxidation only started after uric acid, the main hydrophilic plasma antioxidant, was consumed. In addition, when MeO-AMVN was used as the radical inducer, DCFH oxidation was significantly delayed, indicating its main localization in the aqueous domain.

### Example 4

### Measurement of Plasma Lipid Compartment Oxidation

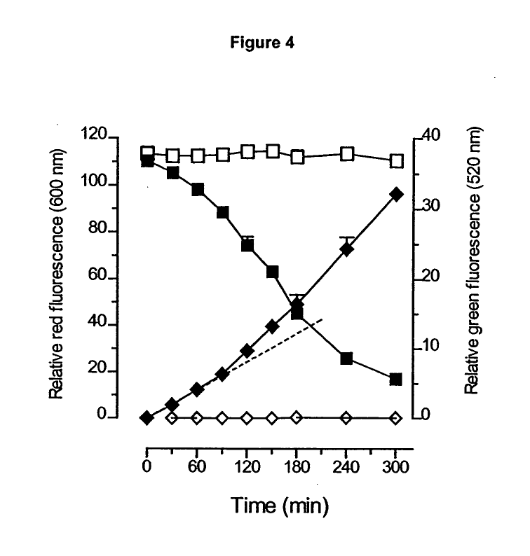

The lipid compartment plasma oxidation was measured using BODIPY, which had been previously found to be a lipophilic fluorescence probe, suitable to monitor the oxidation process in organic solvents and liposomes (Naguib, J. Agric. Food Chem. 48:1150-1154 (2000)) as well as living cells (Pap et al., FEBS Lett. 453:278-282 (1999)). When BODIPY was added to plasma, a linear dose-dependent red fluorescence increase was observed (r²=0.996), indicating the incorporation of the fatty acid analogue in the plasma lipid compartment (data not shown). Only a negligible fluorescence intensity (less than 5-10% with respect to plasma) was observed when BODIPY was added to PBS or a BSA solution (1 g/dl in PBS). Initially, AMVN was used as a typical generator of lipid peroxyl radicals, to induce the oxidative reaction in the lipid compartment. At 2 mM AMVN, there were no observed changes in the BODIPY fluorescence (FIG. 4), probably due to the low efficiency of free radical generation by AMVN in a viscous lipophilic compartment at 37° C. When the concentration of AMVN was increased to 4 mM, a cloudy precipitate formed. Accordingly, a higher efficiency lipophilic radical generator, MeO-AMVN, was used. MeO-AMVN, had a higher efficiency of free radical generation with respect to AMVN (the rate constant is about 15 times larger under the same conditions) (Noguchi et al., Free Rad. Biol. Med. 24:259-268 (1998)).

Results from the lipid oxidation are presented in FIG. 4 which shows time curves of red fluorescence (λex=580 nm, λem=600 nm) and green fluorescence (λex=500 nm, λem=520 nm) of BODIPY in human plasma (1:5 with PBS) in the presence of AMVN and MeO-AMVN. BODIPY red fluorescence: □ (2 mM AMVN), ▪ (2 mM MeO-AMVN); BODIPY green fluorescence: ⋄ (2 mM AMVN), ♦ (2 mM MeO-AMVN). Values are mean±SD of five independent experiments.

When plasma containing BODIPY was incubated in the presence of 2 mM MeO-AMVN, a linear and time dependent decrease of red fluorescence was observed, accompanied by an increase of green fluorescence (FIG. 4). As previously reported (Pap et al., FEBS Lett. 453: 278-282 (1999)), this effect is due to the oxidation of the diene bond with a consequent loss of conjugation between the phenyl moiety and the boron dipyromethen difluoride core which, in isolated form, exhibits a green fluorescence. The green fluorescence increase was significant after 30 min of incubation and increased linearly until 90 min (slope=0.072±0.002 F.U.×min-1). Between 90 and 120 min, we observed a significant change of the slope (0.125×0.004 F.U.×min-1) that correlated with the consumption of α-tocopherol and β-carotene (FIG. 2). No change of BODIPY fluorescence was observed in the presence of 2 mM AMVN or in the absence of the radical initiators for 4 hr (data not shown).

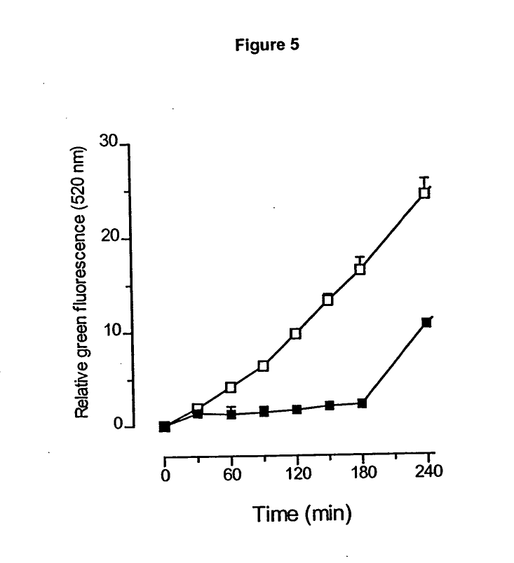

FIG. 5 shows a time-course of BODIPY green fluorescence in human plasma (1:5 with PBS) in the presence of 2 mM MeO-AMVN (□) or 20 mM AAPH (▪). Values are mean±SD of five independent experiments. When 20 mM AAPH was used with human plasma, BODIPY oxidation was delayed 180 min. Oxidation was observed after 240 min, presumably as a consequence of the loss of β-carotene (FIG. 2) and the subsequent initiation of the lipid peroxidation process. BODIPY oxidation began immediately after addition of 2 mM MeO-AMVN. The intra-assay variation of plasma samples in repeated measurements resulted in less than 5% using either fluorescent probe. The CV calculated in the inter-assay precision resulted in 6.4% when DCFH was used and in 8.7% for BODIPY.

To study the lipid oxidation process induced by MeO-AMVN, BODIPY was used as a lipophilic fluorescence probe for the following reasons: (a) it is characterized by a high fluorescence quantum yield limited to the lipid phase, (b) it is stable for several hours in biological fluids at 37° C. (c) it absorbs/emits in the visible region (d) it was found to be a sensitive and selective indicator of lipid oxidation in plasma (e) the initial peroxidation rate is similar to that observed for arachidonic acid (Pap, et al. FEBS Lett. 453:278-282; 1999). Immediately after MeO-AMVN addition, the BODIPY oxidation whose rate constant significantly increased after the depletion of α-tocopherol and β-carotene, whereas it did not appear to be related to the levels of the hydrophilic antioxidants. When AAPH was used as the radical initiator, BODIPY oxidation was significantly delayed suggesting its localization in the lipid phase of plasma, and inaccessibility to the water-soluble peroxyl radicals generated from AAPH.

To measure oxidizability of plasma lipids, a lipophilic radical generator coupled to a selective method capable of detecting lipid peroxidation should be used. The azo-compound 2,2′-azobis(2,4-dimethylvaleronitrile) (AMVN) has been the most frequently used lipid-soluble radical initiator. However, the rate of free radical generation from AMVN is slow under physiological conditions, due in part to a lower efficiency of free radical generation in the viscous lipophilic compartment (Kigoshi, et al. Bull. Chem. Soc. Jpn. 66:2954-2959; 1993). As such, high concentrations of AMVN (20-40 mM) are usually required to induce and sustain the lipid peroxidation process in biological fluids. MeO-AMVN was found to be a suitable lipophilic radical-inducer, since it functioned at concentrations not interfering with the spectroscopic measurement. In contrast, the popular radical initiator AMVN was found to be ineffective at the same concentrations.

### Example 5

### Effect of Plasma Pre-incubation with α-tocopherol and β-carotene on Lipid Oxidizability

To validate the determination of lipid plasma oxidizability and show the protective effect of antioxidants, BODIPY was used as the fluorescence lipophilic probe and MeO-AMVN as the lipophilic radical inducer. The effect of adding the membrane soluble antioxidants, α-tocopherol (in EtOH) and β-carotene (in THF), pre-incubated with plasma, was studied. Both of these fat-soluble antioxidants were found to be effective in protecting the lipophilic probe against radical-initiated oxidation. FIG. 6 shows the effect of time of pre-incubation of human plasma (1:5 with PBS) with α-tocopherol or β-carotene (10 μM final concentration) on lipid plasma oxidizability. Results are expressed as percentage inhibition of BODIPY oxidation induced by MeO-AMVN (2 mM) after 4 hours of incubation. The legend to pattern in FIG. 6 are: blank: no pre-incubation; dotted: 1 hr pre-incubation; lines: 6 hr pre-incubation. Values are mean±SD of five independent experiments. Statistical analysis: one-way ANOVA with Tukey's post test; * p<0.05, ** p<0.01.

The results show a protective effect by adding two lipophilic antioxidants, α-tocopherol and β-carotene to plasma samples. Pre-incubation with these antioxidants improves the enrichment of the plasma lipid compartments where the lipid radicals generated by MeO-AMVN are primarily localized. The protective effect was found to be dependent on the duration of the pre-incubation period, suggesting a slow insertion of α-tocopherol and β-carotene into the lipid compartment when added under in vitro conditions.

Collectively, the results from Examples 2-5 show a fluorescence method to distinguish the oxidizability of the both the aqueous and lipid compartments of plasma, that is characterized by sensitivity, specificity and ease of determination. This method is different from the other conventional methods for measuring total antioxidant capacity, since other methods only measure the aqueous compartment of plasma whereas the present method analyzes both the aqueous and the lipid compartments. This method will be useful in the evaluation of potential antioxidants and in particular to study the lipophilic component of the total antioxidant capacity of plasma.

### Example 6

### (−)-Epigallocatechin-(3)-gallate (EGCG) Protective Effect On Human Plasma Oxidation Induced by Water- and Lipid-Soluble Radical Inducers

This example demonstrated the extent of the protective effect of (−)-Epigallocatechin-(3)-gallate (EGCG) in the aqueous and lipid compartments. To determine the EGCG protective effect on human plasma oxidation induced by water- and lipid-soluble radical inducers, the selective fluorescence method was used to study which plasma compartment EGCG mainly acted as an antioxidant. In particular, the lipid-soluble generator, MeO-AMVN, together with the lipid fluorescence probe, BODIPY, was selected to study the plasma lipid oxidizability. To monitor the aqueous phase oxidizability, AAPH was used as a hydrophilic radical generator and coupled to DCFH was used as the indicator. The amount of free radicals generated by AAPH and MeO-AMVN was kept constant by adjusting the concentration of the two azo-initiators. In the presence of 20 mM of AAPH, the flux of aqueous radicals calculated on the basis of the known rate of free radical generation from AAPH at 37° C., (Rᵢ=1.36×10⁻⁶ [AAPH] mol/liter/sec) (Niki, Methods Enzymol. 186: 100-108 (1990)) was of 2.72×10⁻⁸ mol/liter/sec. To reach the same order of free radicals flux by MeO-AMVN, since the rate of peroxyl radical formation from MeO-AMVN was 14.2×10⁻⁶ [MeO-AMVN] mol/liter/sec (calculated in micelles) (Noguchi et al., Free Rad. Biol. Med. 24 (2): 259-268 (1998)), the concentration of the lipophilic azo-initiator was reduced by 10 fold (2 mM).

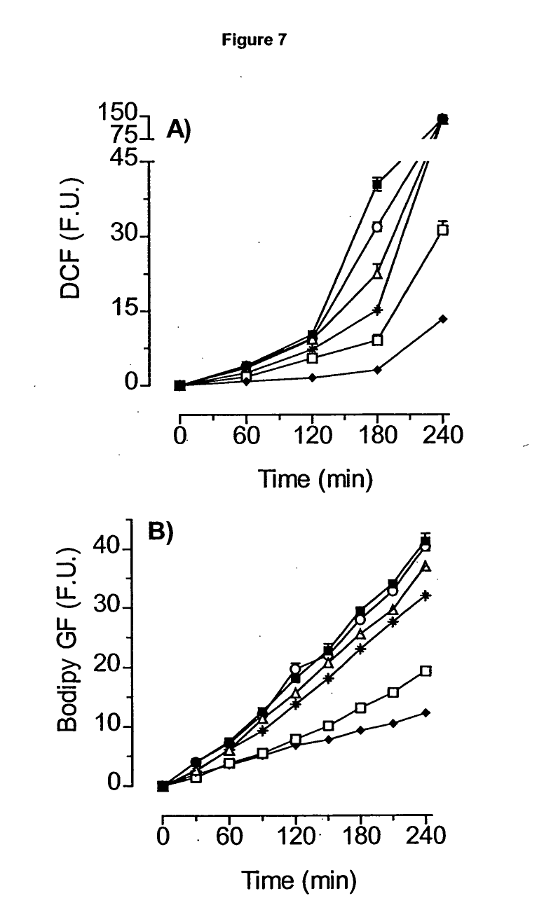

When AAPH was used as radical generator, the aqueous oxidation started after a lag phase of 120 min, corresponding to the depletion of both ascorbic acid and uric acid (Aldini et al., Free Rad. Biol. Med. 31(9): 1043-1050 (2001)). EGCG addition reduced the oxidative process in a dose-dependent manner as shown in FIG. 7A. After 180 min of incubation, EGCG started to be active at 0.25 μM (20.25±0.34%), reaching an almost complete protective effect at 10 μM (93.02±2.02%). FIG. 7A shows that EGCG inhibits aqueous plasma compartment oxidation induced by AAPH (20 mM) and monitored by DCF fluorescence increase (λex=502, λem=520 nm). FIG. 7B shows the EGCG effect on lipid plasma compartment oxidation induced by MeO-AMVN (2 mM) and monitored by measuring BODIPY green fluorescence (BODIPY GF) (λex=500, λem=520 nm). Values are mean±SEM of five independent experiments. ▪ Control; EGCG: (∘) 0.25 μM, (Δ) 0.5 μM, ( [symbol] ) 1 μM, (□) 5 μM, (♦) 10 μM.

When plasma containing BODIPY was incubated in the presence of 2 mM MeO-AMVN, a time-dependent increase of green fluorescence was observed whose rate constant increased following the consumption of α-tocopherol and β-carotene (Aldini et al., Free Rad. Biol. Med. 31(9): 1043-1050 (2001)). The protective effect afforded by EGCG in the lipid domain was found less effective in respect to that found in the aqueous compartment; after 180 min of incubation, the lowest effective concentration was 0.5 μM (13.01±0.56%) and 68±2.3% of protection at 10 μM (FIG. 7B).

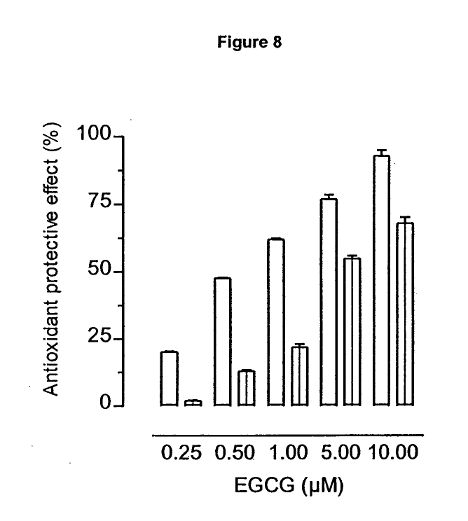

In FIG. 8 the protective effect of EGCG in aqueous and lipid compartments after 180 min minutes of incubation is compared; the calculated IC₅₀ in aqueous and lipid compartments were respectively 0.72 and 4.37 μM. FIG. 8 shows the dose-dependent protective effect of EGCG on aqueous (blank bar) and lipid (filled bar) compartment oxidation after 180 min of incubation. Values are mean±SEM of five independent experiments.

### Example 7

### EGCG Effect on Hydrophilic and Lipophilic Plasma Endogenous Antioxidants Consumption

To show the effect of EGCG on hydrophilic and lipophilic plasma endogenous antioxidants consumption, plasma was incubated with EGCG. When 20 mM AAPH was added to plasma, ascorbic acid and uric acid were almost totally consumed respectively within 15 and 180 min. EGCG at all the concentrations tested (0.5-10 μM) was found ineffective in reducing the consumption of the two hydrophilic endogenous antioxidants (data are not shown). AAPH also induced a significant consumption of lipophilic plasma antioxidants. After 120 min of incubation, the order of consumption expressed as percentage remaining was as follow: α-tocopherol (3.86±0.94)>lycopene (8.49±5.20)>lutein (12.82±4.85)>zeaxanthin (17.50±5.98)≈cryptoxanthin (18.94±3.86)>β-Carotene (28.89±6.17). EGCG addition was found to significantly and dose-dependently reduce the consumption of all the carotenoids (Table 1), indicating its ability to trap aqueous lipid radicals and hence preventing their diffusion into lipoproteins. The sparing effect of EGCG toward α-tocopherol consumption was significant when AAPH concentration was reduced to 10 mM as shown in FIG. 9. The dose-dependent effect of EGCG on α-tocopherol depletion induced by AAPH (10 and 20 mM) and MeO-AMVN (2 mM). The basal content of α-tocopherol was 42.08±1.28 μM. Values are mean±SEM of three independent experiments. * p<0.05 vs control; ** p<0.01 vs. control (ANOVA followed by Dunnett's test) in FIG. 9.

Table 1—Dose-dependent protective effect of EGCG on carotenoids consumption induced by AAPH (20 mM for 120 min) and MeO-AMVN (2 mM for 60 min) in plasma samples. The basal content of carotenoids was as follow: β-Carotene (3.58±0.18 μM); Lycopene (2.10±0.23 μM); Cryptoxanthin (1.77±0.10 μM); Zeaxanthin (0.34±0.01 μM); Lutein (0.72±0.02 μM). * p<0.05 vs control; ** p<0.01 vs control (ANOVA followed by Dunnett's test)

| | | Radical initiator | |
|---|---|---|---|
| | | AAPH | |
| Carotenoid | EGCG (μM) | Residual Amount (%) | MeO-AMVN |
| β-Carotene | 0 (control) | 28.89 ± 6.17 | 50.28 ± 2.26 |
| | 0.5 | 41.51 ± 6.64 | 51.95 ± 1.68 |
| | 1 | 54.38 ± 4.86* | 50.15 ± 3.35 |
| | 5 | 58.63 ± 3.30* | 57.73 ± 0.54 |
| | 10 | 64.35 ± 6.37** | 59.13 ± 3.72 |
| Lycopene | 0 (control) | 8.49 ± 5.20 | 20.12 ± 4.91 |
| | 0.5 | 9.35 ± 5.18 | 20.15 ± 3.22 |
| | 1 | 18.06 ± 2.44 | 19.76 ± 2.62 |
| | 5 | 25.26 ± 1.02* | 23.56 ± 1.78 |
| | 10 | 32.30 ± 4.24** | 26.72 ± 0.94 |
| Cryptoxanthin | 0 (control) | 18.94 ± 3.86 | 41.84 ± 4.24 |
| | 0.5 | 28.46 ± 3.35 | 54.38 ± 2.74 |
| | 1 | 36.21 ± 1.71* | 52.36 ± 0.79 |
| | 5 | 48.88 ± 2.08** | 45.16 ± 3.09 |
| | 10 | 56.45 ± 5.00** | 41.84 ± 1.60 |
| Zeaxanthin | 0 (control) | 17.50 ± 5.98 | 40.04 ± 3.08 |
| | 0.5 | 25.61 ± 6.53 | 47.69 ± 5.85 |
| | 1 | 48.78 ± 4.81* | 49.64 ± 2.14 |
| | 5 | 58.89 ± 5.10** | 44.31 ± 0.94 |
| | 10 | 62.36 ± 11.92** | 42.67 ± 2.04 |
| Lutein | 0 (control) | 12.82 ± 4.85 | 31.04 ± 5.42 |
| | 0.5 | 26.05 ± 4.26 | 32.57 ± 4.14 |
| | 1 | 40.64 ± 5.30* | 33.75 ± 3.94 |
| | 5 | 56.05 ± 4.42** | 44.64 ± 5.50 |
| | 10 | 63.13 ± 8.15** | 40.36 ± 4.03 |

When MeO-AMVN was used to induce a selective oxidation of the lipid compartment, a significant consumption of vitamin E and carotenoids was also observed. EGCG addition was found ineffective in sparing carotenoids depletion at all the concentrations tested (0.5-10 μM) but dose-dependently greatly reduced the vitamin E consumption; the effect was well significant at 1 μM to reach an almost total protection at 10 μM (% α-tocopherol remaining: 96.71±1.46 vs. 16.43±1.72 in control cells; p<0.001).

By using a selective fluorescent method able to induce and monitor the oxidative process in the aqueous and lipid compartments of plasma, the results showed that EGCG dose-dependently protected both aqueous and lipid plasma compartments but with a different potency. The antioxidant efficiency of EGCG was six times greater in the aqueous in respect to the lipid domain (IC₅₀ calculated after 180 min of incubation in aqueous and lipid plasma compartments was respectively of 0.72 μM and 4.37 μM).

EGCG dose-dependently reduced the AAPH induced consumption of the lipophilic antioxidants such as α-tocopherol and polar and apolar carotenoids. FIG. 9 shows the dose-dependent effect of EGCG on α-tocopherol depletion induced by AAPH (10 and 20 mM) and MeO-AMVN (2 mM). The basal content of α-tocopherol was 42.08±1.28 μM. Values are mean±SEM of three independent experiments. * p<0.05 vs control; ** p<0.01 vs. control (ANOVA followed by Dunnett's test). The results show that EGCG, by acting as a radical-scavenger in the aqueous compartment, limits the diffusion of the radical species in the lipid domain, so to prevent the lipid-oxidation cascade and as consequence, the lipophilic antioxidants depletion. By contrast, EGCG was ineffective (up to 10 μM) to spare the main hydrophilic endogenous antioxidants such as ascorbic acid (AA) and uric acid (UA). As reported by Lolito et al. (Lolito et al., Proc Soc Exp Biol Med 225(1):32-8 (2000)), AA acts by preventing catechins depletion and is thermodynamically feasible, in view of the redox potentials [E(EGCG-O., H⁺/EGCG-OH)=0.48 V]; E(A⁻., H+/AH⁻)=0.28 V], to regenerate EGCG from the respective aroxyl radical according to [2]

EGCG-O.+AH⁻→EGCG-OH+A⁻. [2]

With a lesser activity in respect to the aqueous compartment, EGCG was found to dose-dependently inhibit the oxidative damage in the lipid compartment induced by MeO-AMVN. The protective effect can be ascribed to the following mechanisms: (1) EGCG diffuses into LDL where acts as a chain-breaking antioxidant; (2) EGCG binds to the surface of lipoproteins where recycles α-tocopherol from the tocopheroxyl radical.

To understand whether EGCG was able to diffuse inside lipoproteins or remained located to the outer surface of LDL, the sparing effect of EGCG towards α-tocopherol and polar and apolar carotenoids was studied, by using lipophilic peroxyl radicals generated by MeO-AMVN. EGCG at all the concentrations tested (1-10 μM), failed to prevent the depletion of both polar and apolar carotenoids, respectively located in the shell and core of lipoproteins (Borel et al., J Lipid Res. 37(2):250-61 (1996)), while dose-dependently maintained α-tocopherol, which resides at or near the surface of lipoproteins (Kamal-Eldin et al., Lipids. 31(7):671-701 (1996)). These results indicate that EGCG is unable to diffuse in the shell/core of lipoproteins but significantly binds to the outer surface of LDL where the sparing/recycling effect on α-tocopherol can occur. The capacity of EGCG to bind to the outer surface layer of lipoproteins is supported by the affinity of the polar catechin gallates with the polar surface of phospholipids (Carini et al., Life Sci. 67(15):1799-814 (2000); Nakayama et al., Biofactors. 13(1-4):147-51 (2000)) very likely via a complexation mechanism, through electrostatic interactions between the nucleophilic phenol groups of EGCG and the cationic polar heads of phospholipids.

The ability of EGCG to regenerate α-tocopherol was suggested by Jovanovic et al. (Jovanovic et al., J. Am Chem Soc. 117, 9881-9888 (1995)) indicating that EGCG, as well as other green tea catechins, have the required thermodynamic energy (e.g. ΔE=0.06 V at physiological pH) to reduce tocopheroxyl radical and regenerate α-tocopherol according to [3]

Toc.+ArOH→TocH+ArO. [3]

### Example 8

### EGCG Regenerates α-Tocopherol via Reduction of its Phenoxyl Radical: ESR Experiments

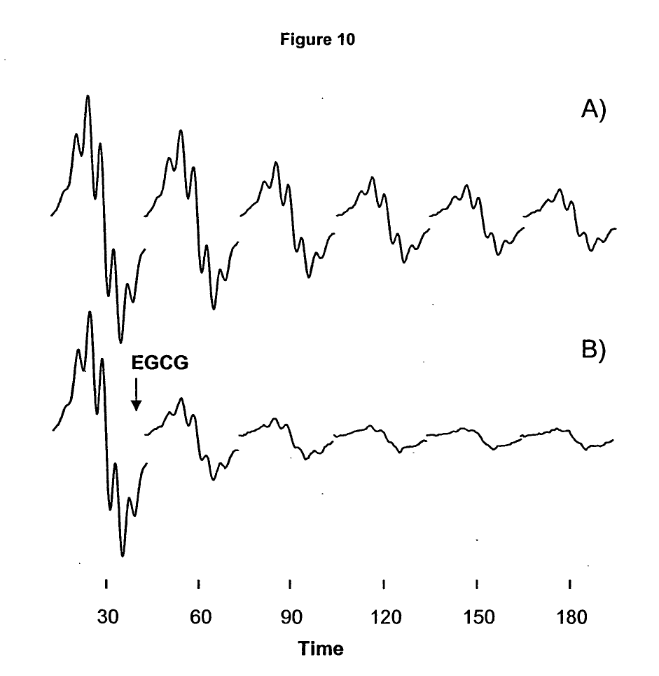

To show that EGCG regenerates α-tocopherol via reduction of its phenoxyl, radical ESR experiments were performed as described in Example 1(vi). 60 sec after mixing α-tocopherol with DPPH, the ESR spectrum of DPPH disappeared completely (due to the scavenging activity of α-tocopherol) and the typical spectrum of α-tocopheroxyl free radical (α-TOC-O.) was observed. In FIG. 10 (panel a) the reported consecutive spectra are displayed (time-intervalled by 30 sec between each other) showing the self-decay of α-TOC-O., described as a second order reaction kinetic by Niki E (Niki E., Methods Enzymol. 186:100-8 (1990)).

FIG. 10 shows an ESR spectra time-course of α-TOC-O. decay in absence (a) and presence (b) of EGCG (15 μM). EGCG addition dose-dependently accelerated the decay rate of α-TOC-O. (FIG. 10, panel b). The quenching effect (calculated after 60 sec the beginning of the reaction) was already significant at 2 μM (% inhibition of ESR signal=8±1.3%) to reach an almost complete disappearance at 25 μM (IC₅₀=12.1 μM). Ascorbic acid, the physiological recycling agent of α-tocopherol showed an IC₅₀=14.2 μM.

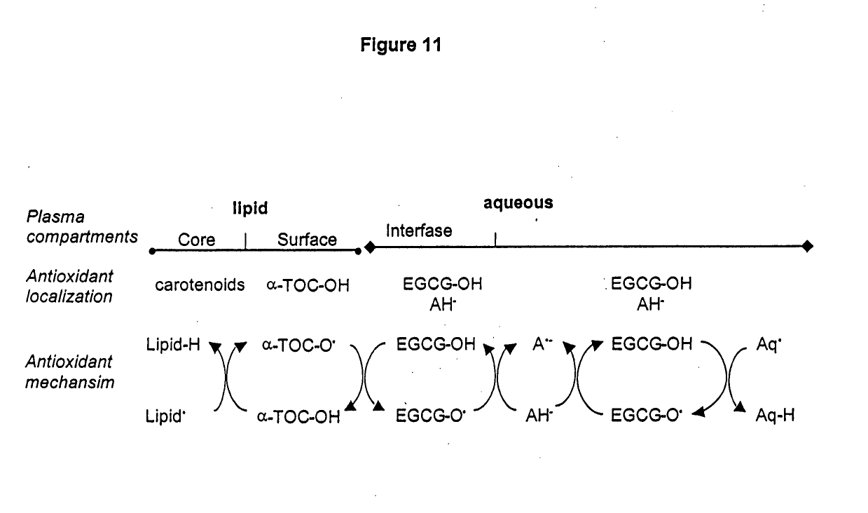

EGCG dose-dependently reduced the AAPH induced consumption of the lipophilic antioxidants such as α-tocopherol and polar and apolar carotenoids. The results indicate that EGCG, by acting as a radical-scavenger in the aqueous compartment, limits the diffusion of the radical species in the lipid domain, so to prevent the lipid-oxidation cascade and as consequence, the lipophilic antioxidants depletion. By contrast, EGCG was ineffective (up to 10 μM) to spare the main hydrophilic endogenous antioxidants such as ascorbic acid and uric acid. Although less than in the aqueous compartment, EGCG was found to dose-dependently inhibit the oxidative damage in the lipid compartment induced by MeO-AMVN. The protective effect can be ascribed to the following mechanisms which is depicted in FIG. 11: (1) EGCG diffuses into LDL where acts as a chain-breaking antioxidant; (2) EGCG binds to the surface of lipoproteins where recycles α-tocopherol from the tocopheroxyl radical. FIG. 11 depicts the proposed antioxidant mechanism of EGCG in human plasma where Aq.=hydrophilic radical species; Lipid.=lipophilic radical species; EGCG-O.=aroxyl radical from EGCG.

To demonstrate the direct reaction of EGCG with tocopheroxyl radical, a direct ESR technique was used. EGCG was found to quench the tocopheroxyl radical with a potency similar to that of AA, supporting the ability of EGCG to regenerate tocopherol through an H-transferring mechanism. This data provides evidence for the regeneration of vitamin E via reduction of its phenoxyl radical by EGCG in LDL particles.

Several previous attempts have been made to demonstrate that GT consumption provides a protection toward LDL oxidation by using isolated LDL and transition metals or AAPH as radical inducers. However, the results have not been consistent. Consumption of six cups per day of green tea or black tea (900 ml/day) for 4 weeks had not significant effect on the resistance of LDL to copper mediated oxidation ex vivo in non-smokers (van het Hof et al., Am J Clin Nutr. 66(5):1125-32 (1997)) or in smoking subject (Princen et al., Arterioscler Thromb Vasc Biol. 18(5):83341 (1998)). In contrast, Ishikawa et al. (Ishikawa et al., Am J Clin Nutr. 1997; 66(2):261-6 (1997)) showed a small but significant prolongation of LDL oxidation ex vivo compared with baseline measurement following 4 weeks of tea consumption (600 ml/day). More recently, Miura and co-workers (Miura et al., J Nutr Biochem. 11(4):216-222 (2000)) found that 300 mg of GT polyphenols ingestion twice daily for 1 week significantly increased the resistance of LDL to ex vivo oxidation. Discrepancy of the results may be due to differences in the experimental procedure as suggested by (Miura et al., J Nutr Biochem. 1 1(4):216-222 (2000)). However, Hodgson (Hodgson et al., Am J Clin Nutr. 71(5):1103-7 (2000)) recently suggested that the lack of effects of tea on LDL oxidation ex vivo might be related to the method used to assess the LDL oxidation. In particular the absence of the protective effect may be due to the isolation of LDL particles from polyphenolic compounds that are mainly present in the aqueous phase of serum. The present data show that EGCG mainly acts as antioxidant in the aqueous in respect to the lipid. In aqueous compartment, EGCG started to be active at 0.25 μM reaching an IC₅₀ at 0.72 μM; these plasma concentrations are easily reachable after and acute/chronic GT supplementation as already reported (Miura et al., J Nutr Biochem. 11(4):216-222 (2000). By contrast, to reach the same order of activity in the lipid compartment, the EGCG concentration needed to be increased by six folds (starting effective concentration 0.5-1 μM, IC₅₀=4.37 μM), a range of concentration more difficult to reach in a controlled supplementation trial.

In summary, EGCG mainly acts as a radical scavenger in the aqueous compartment, preventing the diffusion of the radical process in the lipid domain and consequently sparing lipophilic antioxidants such as α-tocopherol and carotenoids. Under the present experimental conditions, EGCG was unable to diffuse into the lipid compartment and to act as a lipid radical-scavenger. However, EGCG partially inhibited the lipid-peroxidation cascade of the lipid compartment by regenerating α-tocopherol through an H-transferring mechanism. These data suggest that to study the protective effect of GT consumption towards LDL oxidation in ex vivo studies, the usage of whole plasma as substrate coupled to a sensitive method able to monitor the oxidizability of the lipid compartment induced by hydrophilic radicals should be considered.

### Example 9

### Effect of a High Lycopene Diet on Lipid Oxidizability

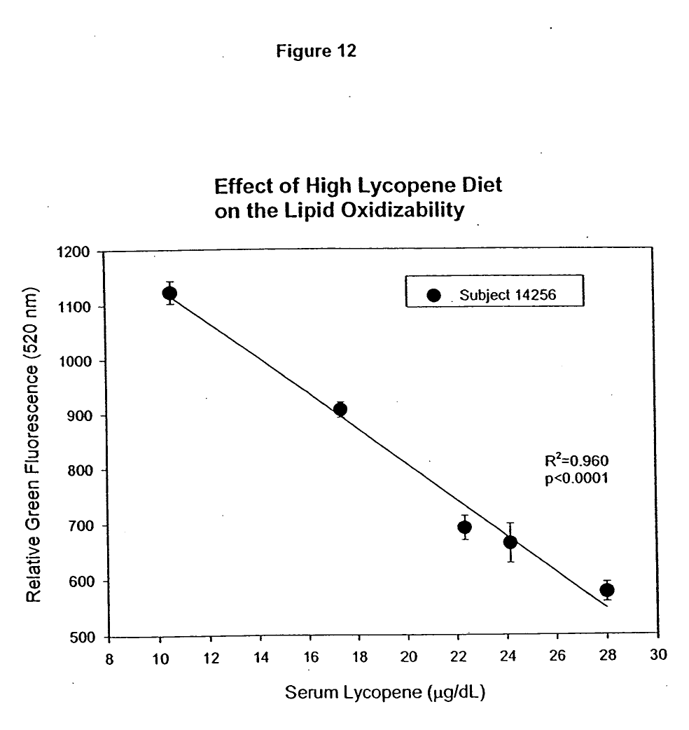

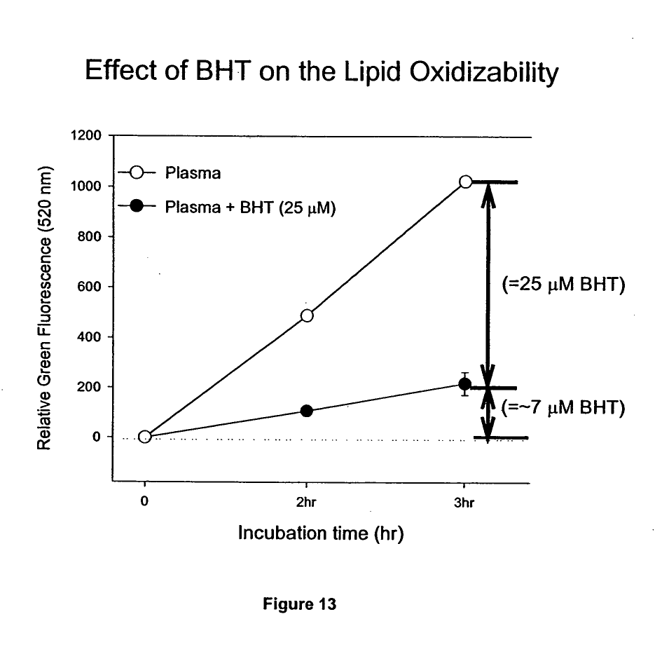

The effects of ingesting antioxidants can now be effectively monitored using the present invention. For example, lycopene, a powerful antioxidant abundant in red tomatoes and processed tomato products, has been linked to the prevention of prostate cancer and some other forms of cancer, heart disease, and other serious diseases. Subjects consumed controlled diets (2-day rotation diet, 10-15 servings of fruits and vegetables/day) with a moderate amount of fat (34% of total energy) for 15 days. Fasting blood samples were collected three times/week and analyzed for carotenoid levels using HPLC and antioxidant capacities in lipid compartment using fluorimetric analysis (MeO-AMVN was used as a radical initiator and BODIPY 581/591 was chosen to monitor oxidation in the lipid compartment). As shown in FIG. 12, plasma lycopene levels were significantly correlated (p<0.0001) with plasma antioxidant capacity in the lipid compartment. This data shows the correlation between a diet rich in lycopenes and reduction of lipid oxidizability, demonstrating their beneficial effects.

### Example 10

### Effect of BHT on Lipid Oxidizability

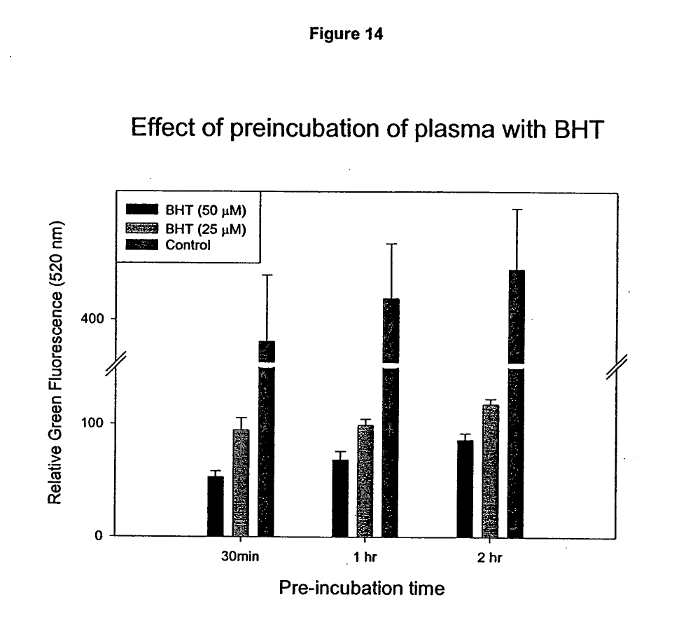

In order to standardize the method to determine the lipophilic antioxidant capacity, butylated hydroxytoluene (BHT), a phenolic synthetic antioxidant, was chosen as an internal standard. Polyenes and certain foods were packaged with added BHT to protect against oxidation. Plasma was incubated with BODIPY 581/591 in the presence and absence of BHT (25 & 50 μM) at 37° C. for 30 min, and determined for oxidizability in the lipid compartment. There was no significant difference among incubation times of 30 min, 1 hr and 2 hr, on the incorporation of BHT in the lipid compartment of plasma (FIG. 14). As shown in FIG. 14, the oxidation of lipid compartment was significantly protected (78%) by BHT and the oxidation of lipid compartment can be expressed as BHT equivalent (32 μM in this subject). The concentration of BHT may also be reduced to a level sufficient to produce a detectable signal. Table 2 also summarizes the results from the study.

The skilled artisan can appreciate that any lipophilic antioxidant which is not present in a subject can be used as internal standard (e.g., carotenoid isomers, synthetic carotenoids, tocopherol isomers, etc.) Antioxidant capacity in the lipid compartment can be expressed as BHT equivalent, BODIPY green fluorescence equivalent (external standard), or other lipophilic standards.

TABLE 2
Antioxidant Capacity in the Lipid Compartment
of Plasma using a BHT Standard

| | Green Fluorescence | | | BHT equivalent |
|---|---|---|---|---|
| Subject # | Plasma | Plasma + BHT | 25 μM | (μM) |

2 hr incubation w/ 1 mM MeO-AMVN (v70L)

| | | | | |
|---|---|---|---|---|
| Subject 1 | 489 ± 21 | 107 ± 1.4 | 382 | 32 |
| Subject 2 | 470 ± 11 | 123 ± 7.3 | 347 | 34 |
| Subject 3 | 387 ± 30 | 142 ± 8.0 | 245 | 39 |

3 hr incubation w/ 1 mM MeO-AMVN (v70L)

| | | | | |
|---|---|---|---|---|
| Subject 1 | 1023 ± 46 | 217 ± 4.0 | 806 | 31.7 |
| Subject 2 | 873 ± 32 | 223 ± 8.9 | 650 | 33.6 |
| Subject 3 | 801 ± 57 | 250 ± 13.3 | 551 | 36.3 |

1. A method for measuring the lipid antioxidant activity in a sample having a lipid compartment and an aqueous compartment, the method comprising:
incubating the sample with a lipophilic radical generator at a concentration that produces free radicals in a lipid compartment of the sample;
adding an oxidizable lipophilic indicator to the sample; and
measuring the oxidation of the lipophilic indicator to provide a measure of the net effects of antioxidants in the sample.

2. The method of claim 1, wherein the step of incubating the sample further comprises incubating a fluid sample selected from the group consisting of blood, plasma, serum, urine, cerebral spinal fluid, amniotic fluid, interstitial fluid, lymphatic fluid, and synovial fluid.

3. The method of claim 2, wherein the sample is plasma.

4. The method of claim 1, wherein the step of incubating the sample with a lipophilic radical generator further comprises selecting a lipophilic radical generator selected from the group consisting of an azo radical generator, and organic hydroperoxide.

5. The method of claim 4, wherein the azo radical generator is selected from the group consisting of 2,2′-azobis(4-methoxy-2,4-dimethylvaleronitrile) (MeO-AMVN), 2,2′-azobis(2,4-dimethylvaleronitrile) (AMVN), azo-bis-isobutylnitrile, 2,2′-azobis(2-methylproprionate) (DAMP), and 2,2′-azobis-(2-amidinopropane).

6. The method of claim 1, wherein the lipophilic radical generator is 2,2′-azobis(4-methoxy-2,4-dimethylvaleronitrile) (MeO-AMVN).

7. The method of claim 1, wherein the step of adding an oxidizable lipophilic indicator further comprises adding an oxidizable lipophilic indicator that is responsive to lipid oxidation.

8. The method of claim 7, wherein the oxidizable lipophilic indicator is a fluorescent probe.

9. The method of claim 8, wherein the fluorescent probe is selected from the group consisting of 4,4-difluoro-3a,4a-diaza-s-indacene (BODIPY) fatty acids, pyrene fatty acid derivatives, perlene fatty acids, cis-parinaric acid, hexadecanamide, diphenyl-1-pyrenylphosphine (DPPP), and lipophilic fluorescein dyes.

10. The method of claim 9, wherein the BODIPY fatty acids are selected from the group consisting of BODIPY 576/589, BODIPY 581/591, and BODIPY 665/676.

11. The method of claim 10, wherein the BODIPY fatty acid is BODIPY 581/591.

12-43. (Canceled)

44. The method of claim 1, wherein the method further comprises incubating the sample with a hydrophilic radical generator at a concentration that produces free radicals in an aqueous compartment of the sample.

45. The method of claim 44, wherein the step of incubating the sample with a hydrophilic radical generator further comprises incubating the sample with a hydrophilic radical generator selected from the group consisting of azo radical generator, 2,2′-azobis[2-(5-methyl-2-imidazolin-2-yl)propane]dihydrochloride, iron, ascorbic acid and metal ions.

46. The method of claim 45, wherein the azo radical generator is selected from the group consisting of 2,2′ azobis(2-amidinopropane)dihydrochloride (AAPH), 2,2′-azobis(4-methoxy-2,4-dimethylvaleronitrile) (MeO-AMVN), 2,2′-azobis(2,4-dimethylvaleronitrile) (AMVN), azo-bis-isobutylnitrile, 2,2′-azobis(2-methylproprionate) (DAMP), 2,2′-azobis-(2-amidinopropane), and 2,2′-azobis[2-(5-methyl-2-imidazolin-2-yl)propane]dihydrochloride.

47. The method of claim 44, wherein the hydrophilic radical generator is 2,2′ azobis (2-amidinopropane)dihydrochloride (AAPH).

48. The method of claim 44 wherein the method further comprises adding an oxidizable hydrophilic indicator to the sample.

49. The method of claim 48, wherein the oxidizable hydrophilic indicator is a fluorescent probe.

50. The method of claim 49, wherein the fluorescent probe is selected from the group consisting of dichlorodihydrofluorescein (DCFH), 4,4-difluoro-5,7-dimethyl-4-bora-3a,4a-diaza-s-indacene-3-propionyl ethylenediamine, hydrochloride, BODIPY FL EDA, and BODIPY FL hexadecanoic acid.
